(19) United States
(12) Patent Application Publication
(10) Pub. No.: US 2006/0084167 A1
(43) Pub. Date: Apr. 20, 2006

(54) Formation of Hybrid Cells by Fusion of Lineage Committed Cells with Stem Cells

(76) Inventors: Menashi A. Cohenford, Huntington, WV (US); John B. Hitz, Boston, MA (US)

Correspondence Address: John B. Hitz, 199 Massachusetts Ave #503, Boston, MA, 02115, US

(21) Appl. No.: 11/163,328

(22) Filed: Oct. 14, 2005

Related U.S. Application Data
(60) Provisional application No. 60/619,510, filed on Oct. 16, 2004.

(51) Int. Cl. C12N 5/06

(52) U.S. Cl. 435/325

(57) ABSTRACT

The potential of a stem cell to differentiate into specialized cell types for restoring normal tissue/organ function has stimulated interest in stem cell research. The methods used to coax stem cells differentiate into specialized cells still remain in their infancy stages. The disclosed invention is the generation of mammalian or avian cell hybrids formed from fusing lineage committed somatic cells with nucleated stem cells or nucleated transit amplifying cells. The fusion of lineage committed somatic cells with nucleated stem cells, or nucleated transit amplifying cells as described herein facilitates stem cell differentiation and lineage commitment of hybrid cells and can be aided by inclusion of an encapsulation step. By the fusion of cells in this invention, this invention also provides for methods to restore damaged tissue or the expression of defective, dysfunctional, decreased, lost or not previously expressed bio-pharmaceutical products.

### CROSS-REFERENCE TO RELATED APPLICATIONS

This application claims the benefit of provisional patent application Ser. No. 60/619,510, filed 2004, Oct. 16, by the present inventors.

There is no federally sponsored research or development and no Microfiche Appendix.

Suggested, U.S. Current Class: 435/346 435/70.2 435/347 435/377 435/378 435/382.

### BACKGROUND OF INVENTION

### Field of Invention

This invention relates to the field of embryology, embryogenesis, molecular and human genetics, human and veterinary medicine, and zoo-technical sciences. This invention relates also to in vitro methods of generating cell hybrids from fusion of (i) terminally differentiated cells or transit amplifying cells, both collectively termed herein as lineage committed somatic cells (“LCSO cells”) with (ii) nucleated adult stem cells, nucleated stem cell-like cells, or nucleated transit amplifying cells (herein after collectively termed “SC cells”), (the hybrid cells hereby termed “LCSOSC cells”). In addition, this invention relates to methods of generating cell hybrids from fusion of (i) nonlymphocytic LCSO cells herein after, “NL-LCSO cells” with (ii) nucleated embryonic stem (nucleated ES) or nucleated embryonic germ (nucleated EG) cells (herein after collectively termed “ESG cells”), (the hybrid cells hereby termed “NL-LCSO-ESG cells”). An additional step(s) of encapsulation before or after fusion allows distinct “conditioning”. This invention additionally relates to (i) the hybrids themselves, and (ii) the restorage of normal function in damaged LCSO and NL-LCSO cells following their fusion with SC and ESG cells, respectively.

Defined Terms and Elaboration Thereof

“Non-immortal”, “mortal” or “not immortalized” cells are defined as cells that cannot be propagated continuously in culture and which innately exhibit a limited number of cell divisions. It is understood that “non-immortal”, “mortal” or “not immortalized” cells also include “self-renewable” cells that cannot be propagated continuously in culture and which exhibit a limited number of cell divisions.

“Immortalized cells” are defined as cells that have been transformed from a finite life span to one possessing an infinite life span, or cells that can be propagated continuously in culture and which exhibit an unlimited number of cell divisions (for example, indefinitely propagates in culture). This definition includes “controlled immortalization” as for example, cells may be transiently immortalized for a controlled period of time (Cheng et al, 2000; Collas et al, U.S. Pat. Appl. No. 20050014258, both incorporated herein by reference).

“Stem cells” are cells with the capacity for unlimited or prolonged self-renewal that can developmentally produce at least one terminally differentiated descendant. Usually, between the stem cell and its terminally differentiated progeny there is an intermediate population of “transit amplifying cells” (precursor cells). The latter cells are lineage-committed, have limited proliferative capacity and are restricted in their differentiation potential. The term “lineage-committed” refers to a situation whereby a cell(s) gets committed to a particular developmental path. Examples of lineage-committed cells are terminally differentiated cells and cells that give rise to tissues such as liver, thyroid (endoderm), muscle, bone (mesoderm), neuronal, melanocyte, epidermal (ectoderm), etc.

“Embryonic stem cells” (ES) are pluripotent cells from the embryo. In other words, they have the ability to give rise to any type of specialized body cell. These cells have the capacity for unlimited self-renewal and can be used for cloning. ES are usually taken from either the morula stage, or the blastula stage (blastocyte stage in mammals) of embryonic development. At the blastocyte stage of development, the early embryo has two distinct structures: an inner cell mass, which will develop into a fetus, and the trophoblast which is an outer ring of cells which becomes the placenta. Removal of the trophoblast from the embryo allows access to the inner cell mass where stem cells are isolated, and extracted. These stem cells may be placed in culture and propagated. Scientists sometimes coaxed these stem cells to promote their differentiation into desired tissue types with “coaxing factors”. If to be used for in vivo transplantation purposes, such specialized and committed cells should preferably be compatible to the recipient's immune system. One way to avoid rejection of these cells by the recipient host is through genetic cloning of the embryonic cells by a procedure known as “somatic cell nuclear transfer”.

“Somatic cell nuclear transfer” is a method whereby nuclear material from an unfertilized ovum (e.g., stem cell) can be removed and the “enucleated ovum” can then be replaced with the nucleus of a somatic cell from the recipient or fused with the recipient's somatic cell.

“Embryonic germ cells” (EG) are pluripotent primordial germ cells that are derived from regions of the fetus destined to develop into testes or ovaries. Like the ES, EG cells grow in culture and have the capacity for unlimited self-renewal.

“Adult stem cells” are nonpluripotent cells. These cells are generally slow dividing, have the ability for prolonged self renewal but yet are typically not immortalized and exhibit plasticity. Being nonpluripotent cells, adult stem cells cannot give rise to any type of specialized body cell. Adult stem cells are found in sites such as the bone marrow [e.g., hematopoietic stem cells (HSC) and mesenchymal stem cells (MSC)], bone marrow stroma, muscle, brain, skin, gut, pancreas, liver and the respiratory tract. Adult stem cells can develop also into transit amplifying cells. This definition of “adult stem cells” also extends to placental, umbilical, fetal derived, or embryonic derived stem cells which exhibit properties of adult stem cells. An example of adult stem cells that can be continuously propagated in culture include adult stem cells that are derived from fecal material described previously by Noninvasive Technologies, Elkridge, Md. (Genetic Engineering News report, 2005).

“Progenitor cells” herein are defined as either (i) adult stem cells and their developmentally downstream “transit amplifying cells” or (ii) the developmentally downstream “transit amplifying cells” only. Although typically not immortal, progenitor cells have been shown to self-renew for many generations in culture sometimes up to 90 generations (Genetic Engineering News report, 2005).

“Side populations” are adult stem cells within tissues that efflux the Hoechst dye 33342 (Hst), (Poliakova et al, 2004).

“Stem cell-like cells” are partially differentiated stem cells or specialized cells in a tissue that de-differentiate (back differentiate) or transdifferentiate. These cells can express earlier stage genes to variable levels. Stem cell-like cells may include “embryonic-like stem cells”, “human ES-equivalent cells”, mesenchymal stem cell-like cells, and neonatal pancreatic duct-derived insulin-producing cells (Basta et al, 2004).

“Transdifferentiation” is defined in relation to developmental plasticity which is a process whereby an adult stem cell or stem cell-like cell from one tissue when present in other tissues or in an environment foreign to itself differentiates into cells of the foreign tissue.

BMCs are bone marrow cells that are classified as “adult stem cells”. Besides serving as a mechanism for normal restorage of blood components, these cells also exhibit two additional important properties 1) a transdifferentiation property, and 2) a hybrid formation (fusion) property (Grove et al, 2004). BMCs can migrate to various tissues and access different organs such as the brain (Crain et al, 2005) and the heart.

It is understood in the aforementioned definitions that every stem cell or stem cell-like cell may change with time upon storage or propagation (Allegrucci et al, 2004). Nevertheless, the term stem cell or stem cell-like cell still applies. This change may include such cases as when pluripotent cells such as ES, EG or stem cell-like cell become restricted in their overall pluripotent capabilities (Orner et al, 2004).

A “somatic cell” is a non germ cell naturally occurring or artificially produced. An example of an artificially produced somatic cell includes a nucleated liposome, a ghost cell into which mitochondria or nuclear material was introduced, or cells that are initiated from cytoplasts (Strelchenko et al, U.S. Pat. Appl. No. 20040259249). An example of somatic cells is “transit amplifying cells”.

“Transit amplifying cells” are lineage committed cells that are in the process of growth and development, in other words, developmentally situated between the less developed adult stem cells and terminally differentiated cells.

A “damaged cell”, damaged tissue or damaged organ represents an entity that is no longer functioning normally or one that has at least one defective, dysfunctional, underexpressed, overexpressed or lost bio-pharmaceutical product(s). Damage (i.e., to cells/tissues/organs) can result from genetic aberrations or maybe physically, biologically or chemically induced, or a combination thereof. Examples of genetic aberrations can include presence of a dysfunctional gene or absence of a gene(s) in general. Examples of physical trauma include ischemic conditions or exposure of a cell/tissue/organ to harmful radiation or extremes of temperature. An example of biologically or chemically induced damage to cells/tissues/organs may result following exposure of the latter to harmful bacterial/viral agents or to toxins. It is hereby understood that “damage” also applies to cells/tissues/organs that have undergone preneoplastic or neoplastic changes.

“Tissue” herein refers to a collection or aggregate of individual cell types.

“Terminally differentiated somatic cells” are non-germ cells incapable of further differentiation.

A “gene product” includes biochemical material either RNA, protein, and derivatives, fragments, analogues or homologues thereof resulting from expression of a gene. A “bio-pharmaceutical product” is defined as a product that helps restore damage (i.e., in cells/tissues/organs) or alleviate the effects of damage.

“Stable hybrid cells” are cells formed as the result of fusing two or more cells and which express a desired bio-pharmaceutical product and/or which perform a desired function.

“Stable hybrid cell lines” are hybrid cells that are continuously grown and propagated in culture.

A “clone” are cells derived from a single cell or common ancestor by mitosis.

“Growth” is increase in spatial dimensions and weight; it may be multiplicative (increase in number of nuclei or cells), auxetic or intussusceptive (increase in the size of cells) or accretionary (increase in the amount of non-living structural matter).

“Differentiation” is the process by which cells become structurally and functionally specialized.

“Imprinting” is a process whereby one of the two copies of a gene within a cell is switched off. In other words, imprinting is the differential degree to which the effects of maternally and paternally derived DNA are exerted.

“Epigenicity” describes any of the mechanisms regulating the expression and/or interaction of genes, particularly, during the developmental process. These include changes that influence the phenotype, but have arisen as a result of mechanisms such as inherited patterns of DNA methylation rather than differences in the gene sequences. “Imprinting” is one such example.

“Cytology” herein refers to the study of origins, structure, function, morphology and/or pathology of a cell or cells. “Cytological examination” or “cytologically examining” herein refers to examining or monitoring a cell or cells for their origin, structure, function, morphology and/or pathology. Preferred cytological examination or monitoring includes cell energy levels (Ishii et al, 2004, incorporated by reference herein) analysis of genomic stability, karyotyping, and genetic analysis. High-resolution analysis of the subtelomeric regions (Darnfors, 2005), and microarray analysis of RNA (Humpherys et al, 2001 and 2002) can for example be used for some of these purposes, (all three references incorporated by reference herein).

“Xenogeneic” herein is defined as in transplantation biology as cells, tissues, or organs originating from different species.

“Syngeneic” herein is defined as cells, tissues, or organs that are of the same species and antigenically the same or similar enough so as not to illicit an immune response, ie., that are histocompatible.

“Allogeneic” herein is defined as cells, tissues, or organs that are of the same species but antigenically distinct.

“Lymphoid” herein is defined as of or resembling lymph or lymphatic tissue including cells that morphologically resemble lymphocytes such as precursors (lymphoblasts) and cells derived from lymphocytes (plasma cells).

“Lymph” herein is defined as, the pale yellow, clear or cloudy fluid that is contained within the vessels of the lymphatic system, and the exudation from a sore.

“Lymphatic tissue” herein is defined as, any vertebrate tissue that is made up predominantly of lymphocytes, e.g. lymph, lymph nodes, spleen, thymus, Peyer's patches, adenoids, pharyngeal tonsils, and in birds, bursa of Fabricus and cecal tonsils.

A “thymocyte” herein is defined as, any lymphocyte found in the thymus. A “splenocyte” herein is defined as, any lymphocyte found in the spleen.

A “lymphocyte” herein is defined as, a leukocyte other than a monocyte or polymorphonuclear cell that is mononuclear and nonphagocytic. Mature differentiated lymphocytes are comprised of B lymphocytes, T lymphocytes and null lymphocytes. After activation the lymphocytes are called large lymphocytes or lymphoblasts, that can then proliferate and differentiate into B and T memory cells, plasma cells, T helper, T suppressor, T contrasuppressor T .sub.DTH, cytotoxic T cells (CTLs), null cells, NK cells, K cells, killer cells, killer T cells. Human lymphocytes in the thymus include early thymocytes with cell surface markers T9 and T10, common thymocytes with cell surface markers T4, T5, T6, T8 and T10, and mature thymocytes with cell surface markers T1, T3, T4 and T10 (helper subset) and T1, T3, T5, T8 and T10 (killer-suppressor subset).

The term “pulse” can include “chemical pulses”, “electrical pulses”, “laser pulses” or “radiofrequency pulses”. The duration of any pulse can be adjusted for an extended period of time.

“In vitro” is defined as biological processes, reactions or experiments that are made to occur in isolation away from the whole organism, for example in a test tube, an artificial environment or in culture.

“Cellular-positioning engineering” is defined as a method of spatially positioning specific cells within the same or other cells. For example, scientists are developing a novel method that integrates the adult stem cell and photolithograph-based, biologic, microelectromechanic system BioMEMS to reconstruct personalized islets (Wang et al, 2004).

### NOTEWORTHY FINDINGS & CONCLUSIONS OF PRIOR ART

The ability of stem cells to differentiate into specialized types of cells and be used to repair damaged tissue/organ has generated great scientific and moral interest in correlated research. Several sources are currently available for the procurement of stem cells. The most common of which include:
(a) Embryonic stem cells, or ES
(b) Embryonic germ cells, or EG
(c) Adult stem cells

Adult stem cells serve as a natural replacement for specialized cells in an adult and have been used in research to avoid the ethical and moral controversies associated with the destruction of ES or EG that are obtained from frozen embryos, embryos deliberately created by in vitro fertilization, or from aborted fetal tissue.

In spite of opposition to their use, arguments exist in support of ES and EG (over the utilization of adult stem cells) that cannot be ignored:
(1) ES and EG are pluripotent and give rise to specialized cells of all tissues, while adult stem cells express limited plasticity and may consequently be less valuable.
(2) ES and EG cells are easier to isolate and propagate; adult stems are few in number and are relatively more difficult to grow in culture.
(3) With age, a decreased number of the adult stem cells exist within a tissue, thus complicating their isolation and exploitation.
(4) The increased susceptibility of adult stem cells with age to genetic mutation has further intensified support for ES and EG.

However, because of the aforementioned ethical controversies, limited access to ES and EG has stagnated work in stem cell research.

For conventional stem cell research to become a reality, extensive research will be needed to effectively and reliably commit stem cells to a particular developmental path.
(1) Intuitively, the more primitive a stem cell is, the more challenging it is to coax the stem cell towards a particular developmental path (i.e., with “coaxing factors”). In other words, for a completely unspecialized cell to become specialized, the cell would likely require more coaxing steps relative to a cell that has already undergone some degree of differentiation (Wu et al, 2004; Wobus and Boheler, 2005).
(2) Likewise, it would be logical to assume that the design and the order of stimuli to differentiate a highly unspecialized cell to a specific cell type would be more difficult to achieve, relative to a cell that already set its path towards a certain specialization.
(3) Though the use of adult stem cells or stem cell-like cells may facilitate this process due to their higher state of development relative to ES and EG, their differentiation to the desired cell type must still be carefully designed.

One proposed and limitedly researched method for the utilization of embryonic stem cells for therapeutic purposes has been the “somatic cell nuclear transfer” procedure (SCNT).
(1) However, while preliminary successes with this approach have been achieved, (Do and Scholer, 2004) this method causes the abnormal re-programming of nuclear material. In studies by Chung et al (2002) and Gao et al (2004), for example, cloned embryonic cells by the SCNT method were found to prematurely express; within the initial few hours of cloning, many somatic cell characteristics which adversely affected the propagation of the cells in culture and their genetic imprinting.
(2) Another problem with SCNT is the high mortality rate of the cloned stem cells which reportedly is greater than 90% (Sutovsky and Prather, 2004).

The direct infusion or transplantation of stem cells in vivo has also encountered limited therapeutic successes (Raff, 2003; Mathur and Martin, 2004).

While it has been previously proposed that myocardium replication and regeneration may occur under conditions of tissue injury by adult bone marrow (due to the marrow serving as a reservoir for cardiac precursor cells), recent studies in mice (Nygren et al, 2004; Balsam et al, 2004; Murry et al, 2004) have proved otherwise, even in instances, where bone marrow cells or hematopoietic stem cells (HSC) were directly injected at the infarct site (Murry et al, 2004).

The development of functional dopaminergic neurons as a result of the grafting of stem cells in animal models for Parkinson's has also proved disappointing. In many instances, recovery was incomplete and the transplanted cells caused the occurrence of teratoma-like tumors (Lindvall, 2001; Love, 2002).

The use of stem cells for myocardial tissue repair has yielded a more promising clinical outcome in humans, but complete, reliable and drastic recoveries have yet to be achieved (Perin et al, 2003; Fuchs et al, 2003; Kang et al, 2004; Mathur and Martin, 2004; Wollert et al, 2004). Moreover, the risk to benefit ratio of the procedures used need still to be assessed. In spite of these shortcomings, the potential value of stem cells is irrefutable. However, for stem cell technology to become a viable therapeutic tool, additional advancements must be made to reliably facilitate the differentiation of cells into specific cell types.

One major finding has been the observation that adult stem cells exhibit a higher degree of plasticity than previously thought. In other words, adult stem cells in one tissue have been shown to migrate to other tissues, giving rise to different cell types beyond their original tissue boundaries (hereinafter referred to as foreign tissue), a process hereby termed as “developmental plasticity” (Eisenberg and Eisenberg, 2003; Raff, 2003; O'Malley and Scott, 2004).

It is imperative to point out that while in certain instances “developmental plasticity” may appear to account for the differentiation of an adult stem cell, the true mechanism for the differentiation may be due to a cell fusion phenomenon; a fusion resulting between an adult stem cell and somatic cells of a foreign tissue (Raff, 2003).

For instance, where it has been reported that the transplantation of hematopoietic stem cells can act as a substitute for hepatocyte transplantation in a murine model of tyrosinemia, (Joshi and Venugopalan, 2004) and HSC transplantation can correct this metabolic liver disease, cytogenic analysis has demonstrated that the true mechanism for this metabolic repair is due to HSC cells fusing with hepatocytes and not due to the “developmental plasticity” phenomenon.

This fusion phenomenon has been also reported to occur between bone marrow derived stem cells with Purkinje neurons and cardiomyocytes forming multinucleated cells (Alvarez-Delado et al, 2003; Nygren et al, 2004).

Another finding has been the utilization of reagents that enhance the immobilization of stem cells to the site of injury. Examples of which include:
(1) Neulasta (M.D. Anderson Cancer Center, drug from Amgen), AMD3100 and G-CSF (granulocyte colony stimulating factor) (NIH) (clinical trial Study ID Numbers: Neulasta (ID03-0164), AMD3100 and G-CSF (040078; 04-H-0078), Nagler et al, 2004).
(2) Additionally reagents such as 5-aza 2′ deoxycytidine, a coaxing factor, have been used to enhance lineage specific differentiation by potentially affecting components of the DNA methylation-silencing system (Allegrucci et al, 2004).

Even though the number of such reagents is on the increase, no specific protocols have been established to reliably prove effective for in vitro and in vivo purposes:
(1) Whereas some reagents or stem cell transfer protocols have proved therapeutically valuable in certain animal models, new studies reveal that the clinical outcome cannot be extrapolated from one animal species to another (Erdo et al, 2004).
(2) Similarly, the outcome of human clinical studies have been shown not to always correlate to observations made with different animal models (Mathur and Martin, 2004).

There have been four recent advances related to ES, adult human pancreatic stem cells and substitutes regarding therapies for diabetes. These four methods (incorporated by reference herein) are different than ours for they do not promote the concept of stem cell hybridization with somatic cells (Tsao et al, U.S. Pat. Appl. No. 20030003088; Habener et al, U.S. Pat. Appl. No. 20030082155; Lumelsky et al, U.S. Pat. Appl. No. 20040121460; and Chan and Kojima, U.S. Pat. Appl. No. 20040132679).

In the mouse and presumably in humans, there are four key developmental stages to producing functional beta cells (Gu et al, 2004):
(1) These start with an unspecified endoderm, then,
(2) pancreatic cells that express Pdx1, then,
(3) by endocrine progenitor cells that express Ngn3, and finally,
(4) by the mature beta cells in the islets of Langerhans.
   Analyses of 3,400 individual pancreatic genes at these four stages have just begun to define these critical development points. Anticipation is to understand the complex endocrine and differentiation mechanisms so as to facilitate the differentiation of ES or other stem cells into becoming functional beta cells (Habener et al, U.S. Pat. Appl. No. 20030082155; Lumelsky et al, U.S. Pat. Appl. No. 20040121460; and Gu et al, 2004).

### SUMMARY & IMPLICATIONS OF THE INVENTION

In the invention disclosed herein, we describe:
(1) In vitro methods for fusing stem cells with lineage committed somatic cells or non-lymphoid lineage committed cells, forming LCSOSC hybrid cells or NL-LCSO-ESG hybrid cells.
(2) Methods for the propagation of said LCSOSC or NL-LCSO-ESG hybrid cells in vitro and approaches including “conditioning” by encapsulation before and/or after fusion, resulting in cells or cellular compositions which can be used for repairing of injured tissues in vivo.

Although the described methods are a compilation of previously reported independent procedures, they are uniquely combined to solve, heretofore, difficult and irresolvable issues.

It has been proposed that a natural system of repair exists in the body that can be overwhelmed by substantial tissue damage (Ye et al, 2003).
(1) The speculation that stem cells may play a role in this natural process of repair has invigorated interest in stem cell research, particularly in restoring normal function to such damaged tissues as the heart, liver, muscle and brain (Raff, 2003; Mathur and Martin, 2004).
(2) The rate of response of stem cells to tissue repair has been attributed to several factors including the availability and number of indigenous stem cells in the damaged tissue as well as the rate of mobilization of migratory stem cells to the injury site (Ye et al, 2003).
(3) Other factors include the mechanism by which different stem cells restore damage (Raff, 2003; Kim, 2004).

The rate of restoration of a damaged tissue by stem cells can therefore be a slow or a fast process as will be demonstrated here using the liver as a model organ.
(1) When injury to hepatic tissue is not severe, the immediate response to damage is effectuated by the native hepatic adult stem cells which serve to replenish the lost hepatocytes in the liver (Ye et al, 2003).
(2) When the extent of injury is severe, hepatic adult stem cells become overwhelmed and the restoration process requires the aid of other stem cells.
(3) Depending on the severity of the injury, oval adult stem cells from surrounding tissue (e.g., the bile ducts) mobilize in the liver and work with the native adult hepatocyte stem cells to effectuate restoration of the damaged tissue (Ye et al, 2003).

Another line of repair includes the BMCs which can also migrate to the liver (Grove et al, 2004). It has been suggested that BMCs effectuate the healing process either by:
(1) Transdifferentiating into new functional hepatocytes, and/or by
(2) Fusing with damaged hepatocytes, in vivo (Wang et al, 2003; Vassilopoulos et al, 2003).

Nevertheless, the BMC mechanism of repair is believed to occur slowly (Wang et al, 2003; Ye et al, 2003).

Is transdifferentiation a real phenomenon? To many investigators it is.
(1) The transformation of BMC cells in the human heart muscle has provided proof that this process occurs in vivo and contributes to the formation of cardiomyocyte like cells in ischemia (Perin et al, 2003; Fuchs et al, 2003).
(2) Nevertheless, in vivo models have yet to dissect which particular stem cells effectuate repair of tissues by transdifferentiation and/or by the fusion phenomenon. Scientists also have lacked the ability to identify which stem cell repair mechanism (transdifferentiation or fusion) accounts for a greater benefit to the tissue restoration process. Unfortunately, these issues have remained unresolved even when animals of the same species (Theise et al, 2003; Hussain and Theise, 2004) were used as an experimental model system. Resulting from this are the following conclusions:
   A better understanding of the physiological role of transdifferentiation and the stem cell fusion process is imperative to assessing the restorative value and potential of stem cells in myocardial and all other tissue injuries.
   Attention must be directed to the efficacy of the methods that are used to coax stem cells with coaxing factors and other methods.

To date different cellular fusion achievements have been described, all incorporated herein by reference:
(1) In U.S. Pat. No. 4,195,125 Adolf Wacker uses polyethylene glycol (PEG) to fuse pancreatic beta cells with Hela cells forming hybrid cells that grew readily in culture.
(2) Methods for fusing pancreatic islet cells with cancer cells (U.S. Pat. No. 4,195,125) and dendritic cells with tumor cells for clinical vaccine applications have been also described (Trevor et al, 2004).
(3) Fusion between different cells has been also achieved by the utilization of lasers (Ohkohchi et al, 2000), electric current (Goding pp. 71-74, 1986) and/or radiofrequency electrical pulses (Chang et al, 1989).
(4) Protocols to effectuate the formation of hybrid cells between very difficult cell partners (i.e., due to such issues as cell size restrictions) have been also described (Chang et al, 1989, Ohkohchi et al, 2000).

How significant is the role of cell fusion in the reparative process? And, do the resultant hybrid cells loose their cellular or molecular identity?

In one recent study, Weimann et al, (2003) transplanted green fluorescent protein-(GFP) bone marrow-derived stem cells in the brain of irradiated mice and demonstrated that the cell fusion phenomenon accounted for bi-nucleated Purkinje neurons that were positive both for calbindin, a Purkinje cell marker and for GFP.

These findings demonstrated for the first time that stable, reprogrammed heterokaryons could be formed in vivo and that the formations of stable bi-nucleate heterokaryons prevented the expression of hematopoietic markers.

It has been proposed that tetraploid cells formed as a result of fusion have greater functional capacity than the equivalent cell mass of diploid cells. This enhanced functionality has been attributed to:
(1) Their overall increased mRNA content (Rice and Scolding, 2004).
(2) Their higher energy levels as a result of them not having undergone mitosis.

BMC-derived revitalization of tissue by fusion in which higher ploidy levels occur has been suggested to benefit from this increased functionality. Increased functionality has been demonstrated to occur in tissues such as the liver, muscle, heart and brain including Purkinje neurons (Rice and Scolding, 2004) where the cells typically undergo homotypic fusions.

Once fusion occurs (i.e., rendering the cells polyploidy), it has been suggested that reduction division takes place, restoring the resultant hybrid cells to their normal diploid state. The successful grafting of bone marrow cells to liver cells and of brain cells to bone marrow cells is evidence supporting this hypothesis (Rice and Scolding, 2004).

Epigenetic block-modulation is not an unusual occurrence and has been demonstrated both in vitro and in vivo.

Kohler and Milstein (1975, 1976) fused a murine B cell with a murine myeloma cell and found that the resultant hybrid was producing antibody and was continually growing in culture.

Similarly, human antibody producing hybridomas have been generated between human lymphocytes and such immortalized human cells as lymphoblastoid B, immortalized B cells and malignant melanoma cells, in vitro (U.S. Pat. No. 4,761,377; U.S. Pat. No. 5,126,253; U.S. Pat. No. 6,051,229).

Fusion between human dendritic cells and tumor cells has resulted in hybrid cells retaining the antigen-presenting and the immune-stimulatory capacities of the dendritic cells (Trevor et al, 2004).

In another study, Hela cells were fused with pancreatic cells defective in their insulin production and the resulting hybrids were found to yield insulin in response to a glucose stimuli (U.S. Pat. No. 4,195,125).

Other examples of epigenetic block modulation include the in vivo fusion of HSC cells with cardiomyocytes and the observation of striated cardiomyocytes type of cells which resulted from the fusion (Nygren et al, 2004), incorporated herein by reference.

Historically, the initial cell fusion studies were carried out with chemicals and were restricted to hybridoma research utilizing murine cells (U.S. Pat. No. 4,761,377). Shortly, thereafter, successful fusion of human cells to form stable human hybridoma cell lines was also achieved (U.S. Pat. No. 4,916,072), both references incorporated herein by reference.

Methods to chemically fuse lymphocytes and immortalized cell types, including lymphoblastoid B cells, immortalized B cells and malignant melanoma cells are well known in the art (U.S. Pat. No. 4,761,377; U.S. Pat. No. 6,051,229; U.S. Pat. No. 5,126,253; Kohler and Milstein 1975 and 1976; Gefter et al. 1977; all incorporated herein by reference).

As mentioned earlier, our knowledge and protocols to date for allowing stem cells to differentiate are limited:
(1) Coaxing methods with coaxing factors are still in their infancy stages of development (Barberi et al, 2005).
(2) Clinical studies have yet to establish the correct sequence of events to effectuate the development of stem cells to a particular path.
(3) Developing a clearer understanding of the transdifferentiation phenomenon has been also hampered by the fact that only a few cells undergo the transdifferentiation process in vivo and, when this occurs, transdifferentiation accounts for only marginal functional improvements (Perin et al, 2003; Fuchs et al, 2003; Cogle et al, 2004, Mathur and Martin, 2004).
(4) The fusion of stem cells with other cells, in vivo, has been also very difficult to prove (Medvinksy and Smith, 2003) as has been assessing the role of this process for reparative purposes.
(5) Methods to demonstrate the fusion of stem cells with other cells in a living system have been also technologically lacking (O'Malley and Scott, 2004).

We here propose a novel approach for:
(1) Reliably coaxing stem cells to differentiate into desired cells types, and
(2) For allowing the formation of an increased numbers of lineage committed cells to effectuate repair.

How then is our approach different than what has been conventionally practiced?

Uniqueness of Approach

It has been accepted by many investigators that the in vivo fusion of stem cells with other cells offers no therapeutic value and fails to provide a viable tool for reparative purposes (Mathur and Martin, 2004). The low frequency of stem cells fusing with other cells in vivo (Mathur and Martin, 2004) could readily explain why investigators have overlooked or under played the therapeutic benefits of this phenomenon. Based on the examples provided herein:
(1) A strong argument is made in support of the therapeutic values that can be derived from fusing stem cells with other cells.
(2) claims are made in this invention that pull us away from the popular belief that fusion between stem cells and somatic cells offer limited therapeutic value.

Assertions are also made that epigenetic block modulation would allow all herein claimed hybrid cells to commit to a development path allowing specific types of cell to be made for various therapeutic purposes.

As a means to increase the number of all herein claimed hybrid cells relative to their natural frequency of formation, in this invention emphasis is made also on:
(1) In vitro methods for fusing multipotent stem cells (nucleated adult stem cells, nucleated stem cell-like cells) or nucleated transit amplifying cells (collectively, “SC cells”), or nucleated ES or nucleated EG cells (collectively, “ESG cells”) with lineage committed somatic cells and the propagation of the resultant hybrid cells in culture.
(2) In contrast to common practices, where the healing of the tissue is left to few hybrid cells that are formed naturally, this invention relies on making a large number of any herein claimed hybrid cells; i.e., that are prepared and grown in vitro quickly available to different damaged tissues.

The large doses of all herein claimed hybrid cells at the site of damage should provide an efficacious and rapid approach to overcoming the slow natural repair process caused by the in vivo stem cell fusion phenomenon.

While the in vitro fusing of “immortalized” stem cells with other cells for reparative purposes has been proposed (Young, 2004), incorporated herein by reference, ‘immortalized’ hybrid cells may grow out of control if given to a patient for therapeutic purposes. Moreover, no methods were described for performing the fusions.

One proposed approach to avoid the continuous in vivo growth of “immortalized” stem cell hybrids is having to use suicide genes. If the fusion occurs with cells that are more prone to naturally becoming cancerous then the use of a suicide gene is preferred and selected for in the selection process. A potential problem with this approach, however, is the possibility that:
the suicide gene may fail to function, for instance, due to genetic instability or gene translocation issues, and/or
the hybrid cells may become cancerous.

Proposed methods for effectuating fusion of immortalized stem cells with other cells all incorporated herein by reference include:
(1) The use of “conventional fusion technique(s)” (herein defined as chemical treatment methods or viral methods) described herein and more preferable among the chemical treatments, the utilization of PEG.
(2) The use of electricity as described (e.g., Goding pp. 71-74, 1986).
(3) The use of radiofrequency pulses as described (e.g., Chang, D. C., 1989).
(4) The use of lasers as described (e.g., Ohkohchi et al, 2000).
(5) The use of “natural fusion technique(s)” as described (Tomczuk et al, 2003) herein incorporated by reference. Natural fusion techniques are defined as those being promoted, for example by cell surface molecules that promote fusion between two cells. Overexpression of beta integrin in one cell and the ADAM protein disintegrin domain in another cell could for instance stimulate such fusion.

Most efforts to date have relied on the direct coaxing of stem cells in vivo.
(1) One example of such coaxing methods has included the placement of stem cells in a microenvironment (i.e., tissue) foreign to where they were originally isolated with the hope of inducing the cells to become lineage committed to specific cell types common to the injured tissue. The in vivo tissue and its microenvironment are the coaxing factors.
(2) Other examples have included the use of chemical stimulants to induce the mobilization of stem cells to different body sites in the anticipation that once at the site, the cells would transdifferentiate or fuse with other cells to effectuate repair.

Although scientists have placed a greater emphasis on the use of pluripotent stem cells such as ES and EG for the treatment of damaged tissue in part because they are immortal, in recent years, there has also emerged an increased interest in the use of adult stems for reparative purposes.

Reasons for this interest have been:
(1) Evidence supporting the developmental plasticity of the cells and
(2) The suggestion that adult stem cells can fuse with damaged cells in vivo, forming hybrid cells that are capable of repairing damaged tissue.

Nevertheless, as stated earlier, conclusive evidence to support this hypothesis has yet to be provided (O'Malley and Scott, 2004).

The fusion of an adult stem cell with damaged somatic cells of a tissue makes intuitive sense if one plans to restore functionality in damaged cells.
(1) By virtue of epigenetic dominance, the fused non-immortal stem cell is likely to acquire the cellular and molecular identity of the damaged cells so as to also acquire full functionality.
(2) Once fusion occurs, rendering the cells polyploid, reduction division causes the resultant hybrid cells to undergo a normal diploid state.
(3) Being more differentiated relative to ES and EG, an adult stem cell is also likely to require less of a coaxing than ES or EG when induced to transdifferentiate or undergo the fusion phenomenon.
(4) Having a limited self-renewal capability, a non-immortal stem cell is also likely to be a better candidate for human therapy than the ‘immortal’ ES and EG.
(5) By in vitro fusing of a non-immortal stem cell with a damaged somatic cell and the subsequent propagation thereof to a limit, a large amount of hybrid cells can be made available in vivo thus allowing for a quick determination of the therapeutic role of the fusion phenomenon in tissue repair.

Methods to monitor the expression of specific gene products and the in vitro regulation of specific products in hybrid cells are known in the art (Mathur and Martin, 2004; O'Malley and Scott, 2004) both incorporated herein by reference. Additionally, various methods to effectively fuse mammalian and in particular human cells have been described (Kohler and Milstein, 1975 and 1976; Goding pp. 71-74, 1986; Chang, D. C., 1989; Ohkohchi et al, 2000).

In one preferred embodiment: The use of methods for fusing stem cells with other cells in vitro is described herein.

In a second embodiment: The propagation of all herein claimed hybrid cells in culture and the monitoring thereof to assess fusion and functionality is proposed.

In a third embodiment: Methods to determine which hybrid cells offer the best therapeutic potential are described.

In a fourth embodiment: Methods for introducing these hybrid cells in vivo are elaborated.

With the non-immortal stem cells being able to regenerate to some extent in vitro (Zeng et al, 2004), the propagation of all herein claimed hybrid cells would provide for a source of reparative functional cells. Although said LCSOSC hybrid cells are typically not immortal, progenitor cells have been shown to self-renew for many generations in culture and sometimes up to 90 generations (Genetic Engineering News report, 2005). In instances where propagation of claimed hybrid cells is required to be extended, “controlled immortalization” (cells transiently immortalized for a controlled period of time; Cheng et al, 2000; Collas et al, U.S. Pat. Appl. No. 20050014258) is preferred.

Human adult stem cells and human ES (hES) cell lines have been shown to be genetically stable for the periods required for their in vitro or ex vivo expansion. Spontaneous cancerous transformation of human adult stem cells in vitro was first reported in April, 2005, but only after long-term in vitro propagation in culture (i.e., 4-5 months versus the typical ex vivo expansion period of 6-8 weeks—See Rubio et al, 2005). Methods to assess genomic stability during in vitro propagation have relied on such techniques as high-resolution analysis of the subtelomeric regions which allows for the sensitive detection of alterations in base pairs (Darnfors et al, 2005) incorporated herein by reference. For purposes of this invention, high-resolution analysis of the subtelomeric regions will hence be used as the preferred cytological examination method.

Furthermore, to avoid hybrid partnering between cancerous cells and stem cells the fusion of cells described herein can be made to be selective. To date, to the knowledge of the authors, there have been no known cases of cancer arising from the injections of adult stem cells in any animal (Levesque, 2004). Since fusion of injected adult stem cells with a patient's cells may occur naturally, had cancer been an issue, it would have already been observed in human clinical trials. As it is, epigenetic block-modulation from cellular fusion using adult stem cells seems to be developmentally far enough downstream to prevent cancer. Nevertheless, as a precautionary measure, this invention allows for, resultant fused cells to be screened by high-resolution analysis of the subtelomeric regions for genetic stability after both in vitro or ex vivo propagation. These concepts extend to cells developmentally equal or downstream of stem cells, including nucleated stem cell-like cells, and nucleated transit amplifying cells.

In vitro fusion can be also performed in the presence of different coaxing factors to yield the desired specialized cells for reparative in vivo purposes. The combination of these embodiments provides for a novel method to facilitate stem cell research and allows for:
(1) Solving many irresolvable issues,
(2) Introducing modifications and concepts not suggested in prior art,
(3) Succeeding where others have failed to provide sustained patient therapeutics,
(4) Realizing advantages that never before were appreciated,
(5) Succeeding in the implementation of prior art where others have failed to perceive its value in stem cell research, (i.e., due to concepts described herein contrary to the teachings of the prior art in the field), and
(6) Designing an operative solution where before failure prevailed.

Procedures for the isolation of stem cells (Thomson et al, 1995 and 1998; Uchida et al 2000; Turnpenny et al, 2003; Zeng et al, 2004; Messina et al, 2004)), porcine pancreatic cells (U.S. Pat. No. 4,195,125), human islet cells (Matsumoto et al, 2004a, 2004b), human pancreatic cells (Bugliani et al, 2004), small animal islet (Gurol et al, 2004), and somatic cells including murine adult pancreatic stem cells (Seaberg et al, 2004), human hematopoietic progenitor cells (HPCs) (Hart et al, 2004), BMCs (Nakamura et al, 2004), neural progenitor cells (Luo et al 2003), human fetal neural progenitor cells (Tamaki et al, 2002), human umbilical cord mesenchymal stem cell-like cells (Romanov et al, 2003), cardiomyocytes (Piper and Isenberg, 1989), hepatocytes (Berry and Friend, 1969; Overturf et al. 1999), neurons (Alvarez-Dolado et al, 2003), epidermal (Hematti et al, 2002) and endothelial cells (Joyce and Zhu, 2004) are well known in the art and all incorporated herein by reference.

Disease-Related Applications of Proposed Procedures

The invention as described herein specifically relates to:
(1) Hybrid cells, or
(2) Hybrid cell lines formed as a result of fusing a mixture of lineage committed somatic cells (hereby termed “LCSO cells”) and SC cells. The SC cells are chosen from a group consisting of non-immortal or immortal:
   Nucleated adult stem cells,
   Nucleated stem cell-like cells, and
   Nucleated transit amplifying cells.
(3) The use of LCSOSC hybrid cells in the biological classes of mammals, or aves, preferably in mammals, most preferably in humans, for therapeutic purposes. The use of LCSOSC-containing hybrids in this invention can encompass the mixture of LCSO, SC cells and LCSOSC cells or the selected LCSOSC cells or subset(s) containing hybrid cells therefrom, hereby termed, “collective SC mixture”.
(4) Similarly formed hybrids between NL-LCSO and ESG cells and use thereof are described.

In one preferred embodiment: A disease condition/tissue injury can be repaired by infusion of any of herein claimed hybrid cells or combinations thereof at the site of injury which would then by virtue of its limited propagation properties (i.e., in vivo) effectuate repair of appropriate target tissue/organs, more preferably in mammals and most preferably in humans.

In another embodiment: The introduction of any of herein claimed hybrid cells or combinations thereof can be achieved distant from the site of injury or diseased tissue. Cases where these embodiments would solve an immediate need include treatment of conditions such as: Alzheimer's disease, amyotropic lateral sclerosis, birth defects, bone replacement, cancer, cartilage replacement, Crohn's fistula, diabetes, epilepsy, glomerular disease, hair replacement, heart disease, kidney disease, ligament replacement, liver disease, lung disease, multiple sclerosis, muscular dystrophy, Parkinson's disease, renal ischemic disease, senile dementia, severe burns, skin disease, spinal cord injuries, stroke and tooth replacement.

Other conditions that can also benefit from these embodiments include: Bone-marrow deficiency diseases and certain diseases of the eye such as macular and retinal degeneration and in situations involving retinal detachments.

Therapeutic benefits from these embodiments are also realized in certain metabolic and genetic disorders examples of which include: Addison's Disease, Tay-Sachs disease, Gauchers disease, Maple Syrup disease, Phenylketonuria, Galactosemia, Sickle cell anemia, Thalassemia, Hemophilia, alpha-1 antitrypsin deficiency-emphysema and Tyrosinemia.

Of the various diseases that in part can be therapeutically treated by the invention herein—diabetes, heart disease and Parkinson's will be elaborated as a model system.

With the human pancreas just recently identified with tissue specific adult stem cells (Levine and Mercola, 2004; Habener et al, U.S. Pat. Appl. No. 20030082155, both references incorporated herein by reference), tissue reparation of the diabetic pancreas faces new challenges in stem cell research. The differentiation of human pancreatic adult stem cells, by coaxing factors in vitro into either a beta cell, an alpha cell, a pseudo-islet like aggregate, or a hepatocyte has recently been accomplished (Habener et al, U.S. Pat. Appl. No. 20030082155) however, safety in regards to cancer (genomic stability), regulation of glucose metabolism (balanced regulation of hormones between the various pancreatic cells), and side effects (eg., liver toxicity) through time have yet to be analyzed.

The interactions between insulin (beta cells), glucagon (alpha cells) and somatostatin (delta cells) in particular must be well understood if one is to chart a curative approach for the disease. Attention must be devoted not only to understanding the rates and factors which influence the secretion of these endocrine hormones, but also to the regulation of cell energy levels during normalcy and stress.

In one preferred embodiment: Patient's pancreatic islet cells are fused in vitro with patient's bone marrow cells consisting of hematopoietic progenitor cells (non-immortal HSCs and their corresponding transit amplifying cells and side populations) and mesenchymal progenitor cells (non-immortal MSCs and their corresponding transit amplifying cells and side populations).

In another preferred embodiment: Fusion of pancreatic cells is carried out with specific non-immortal bone marrow cells, namely, the hematopoietic progenitor cells.

Alternatively, fusion of pancreatic islet cells in this invention herein can be performed with human pancreatic adult stem cells, the latter recently found to exist (Habener et al, U.S. Pat. Appl. No. 20030082155).

In another embodiment: Patient's pancreatic islet cells are fused in vitro with a donor's bone marrow cells consisting of hematopoietic progenitor cells (non-immortal HSCs and their corresponding transit amplifying cells and side populations) and mesenchymal progenitor cells (non-immortal MSCs and their corresponding transit amplifying cells and side populations). In this embodiment fusion of pancreatic cells is preferably carried out with specific bone marrow cells, namely, the hematopoietic progenitor cells.

In another embodiment: Pancreatic islet cells are taken from cadaveric human donor pancreata, fused in vitro with the patient's bone marrow cells consisting of hematopoietic progenitor cells (non-immortal HSCs and their corresponding transit amplifying cells and side populations) and mesenchymal progenitor cells (non-immortal MSCs and their corresponding transit amplifying cells and side populations). In the preferred embodiment fusion of pancreatic cells is carried out with specific bone marrow cells, namely, the hematopoietic progenitor cells.

Other potential cell fusion partnerships include the use of beta pancreatic cells from cadaveric pancreata with donor bone non-immortal bone marrow cells or the use of beta pancreatic cells from pancreata of living donors and donor non-immortal bone marrow cells.

Where examples are not provided, it is understood that the source of non-immortal bone marrow cells can be from cadaveric tissues, living donors or from a patient with the disease.

It is also understood that fusion of cells in all the instances in this invention can be performed in the presence of a coaxing factor(s) in aqueous media and/or in media supplemented with organic solvents. A coaxing factor(s), includes such reagents or molecules as 5-aza 2′ deoxycytidine, a growth factor or an antibody neutralizing agent.

The limited propagation of hybrids in this invention (i.e., due to the non-immortality of the stem cells) can be performed by growing the cells in standard or specifically designed growth media with or without serum supplementation. Examples of tissue culture media are known in the art (Freshney, 2000), the book incorporated herein by reference.

The current treatment of cardiac ischemia and infarction with BMC transplantations is believed to occur by the BMCs releasing angiogenic factors that have been shown to stimulate cardiac growth and repair only marginally.

Stem cell fusion with cardiomyocytes in vivo has been postulated to occur rarely and to be of no or minimal therapeutic value (Perin et al, 2003; Fuchs et al, 2003; Kang et al, 2004; Wollert et al, 2004, all incorporated herein by reference).

In one embodiment: Intracoronary muscle wall injections are used for introducing the hybrid cells described herein in the myocardium.

In another embodiment: In vivo cardiac treatment is proposed by injecting, at the site of injury, in vitro fused non-immortal BMCs with cardiomyocytes that were propagated in culture.

In another embodiment: In vivo cardiac treatment is proposed by injecting, at the site of injury, in vitro fused non-immortal HSCs with cardiomyocytes that were propagated in culture.

In another embodiment: In vivo cardiac treatment is proposed by injecting, at the site of injury, in vitro fused non-immortal MSCs with cardiomyocytes that were propagated in culture.

In a preferred embodiment: Intracoronary muscle wall injections are used for introducing the hybrid cells described herein in the myocardium.

In another embodiment: Intracoronary muscle wall injections of the hybrid cells described herein are performed in the myocardium using a cocktail of one or more coaxing/growth factors.

In another embodiment: Intracoronary muscle wall injections of the hybrid cells described herein are performed in the myocardium in the presence of unfused stem cells.

In another embodiment: Cardiomyocyte sheets derived from hybrid cells as described herein are proposed for cardiac graft implants.

In another embodiment: Extracoronary injection of encapsulated implants containing one or more types of hybrid cells of this invention (e.g., HSC, MSC and/or the BMC based) are performed in the myocardium.

In another embodiment: Extracoronary injection of encapsulated implants containing one or more types of hybrid cells of this invention (e.g., HSC, MSC and/or the BMC based) are performed in the myocardium using a cocktail of coaxing and/or growth promoting factors.

It is understood in all the embodiments relating to the treatment of the heart that polymorphonuclear cells maybe excluded.

Parkinson's disease is another candidate for treatment by this invention. Parkinson's disease is due to the degeneration and death of dopaminergic neurons in the substantia nigra.

In a preferred embodiment: The treatment of Parkinson's is proposed to occur by transplanting in vitro generated hybrid cells formed from fusing non-immortal stem cells with neuronal cells.

In another embodiment: The treatment of Parkinson's is proposed by transplanting in vitro generated hybrid cells formed from fusing non-immortal stem cells and neuroglial cells.

In another embodiment: The treatment of Parkinson's in proposed by transplanting in vitro generated hybrid cells formed from fusing non-immortal stem cells and oligodendrocytes.

In another embodiment: The treatment of Parkinson's is proposed by transplanting in vitro generated hybrid cells formed from fusing non-immortal stem cells with any combinations of neuronal, neuroglial and oligodendrocytes.

It is understood that the in vitro propagation of hybrids of this invention may yield neurospheres which are preferred for the treatment of Parkinson's. “Neurospheres” are defined as multicellular spheres of cells containing neurons and astrocytes in addition to neural stem cells. In culture neurospheres can quickly increase in size and generate a large numbers of neural stem cells (Kim, 2004), incorporated herein by reference.

It is also understood that grafting methods can be performed by encapsulating the various combination of the hybrid cells described herein for Parkinson's.

One general method of this invention for treating patients is to acquire and use patient sample as described herein. The targeted LCSO cell or NL-LCSO cell is obtained, diseased or not, from the patient, mixed and fused with most preferably syngeneic or less preferably allogeneic non-immortal SC cells or ESG cells, to provide any herein claimed hybrid cells. Specific herein claimed hybrid cells are then grown in culture, examined cytologically, (i.e., including evaluated for desired functionality) then provided to the patients in the form of graft or injection, etc. Alternatively, optional selection of any herein claimed hybrid cells (selected hybrids) can be performed following their growth, morphological examination and determination of functionality in vitro. The selected hybrids can then be provided to the patients in the form of graft or injection, etc.

Another general method of this invention for treating patients is to acquire and use patient sample as described herein. The targeted LCSO cell is obtained, diseased or not, from the patient, mixed and fused with most preferably syngeneic or less preferably allogeneic immortalized SC cells, or ESG cells to provide herein claimed hybrid cells. Specific herein claimed hybrid cells are then grown in culture, examined cytologically, (i.e., including evaluated for desired functionality) then provided to the patients in the form of graft or injection, etc. Alternatively, optional selection of any herein claimed hybrid cells (selected hybrids) can be performed following their growth, morphological examination and determination of functionality in vitro. The selected hybrids can then be provided to the patients in the form of graft or injection, etc. It is understood that the preferences for embodiments provided for non immortal stem cells, including antigenicity, apply also for immortalized stem cells.

The most preferred use of syngeneic cells ensures that no major adverse immune responses are elicited to the graft or the injected material when provided to the same patient.

In this invention, any herein claimed hybrid cell(s) as provided herein can be used also to grow distinct tissue types, such as muscle, blood, vessels, cartilage, bone, neurons, and islet cells. When syngeneic cells are used for fusion purposes, no immunological match is required. In this invention, any herein claimed hybrid cell(s) can be made recipient-independent and with broad range of applicable utility.

Alternative to syngeneic applications this invention also teaches the art of encapsulating any herein claimed hybrid cell(s) for in vivo transplantation as a means for escaping patient's immune system.

“Encapsulated cell technology” is when a cell or a group of cells are encapsulated, that is to say, are held within or are coated by a gel or polymer layer, examples of such layers include: alginate, gelatin, albumin, truncated albumin, albumin fusion proteins, fibronectin, or semi-permeable polymers (Ruel-Garie'py and Leroux, 2004, incorporated herein by reference). Other encapsulated cell technologies include all medical devices that allow sequestering of cells from the surrounding environment. One example, of such devices are hollow fibers. It is also understood that encapsulation can be accomplished by capturing cells on a surface such as a microbead, microparticle, microcarrier, nanocarrier or nanoparticle followed by coating of the cells on the surface such as another layer(s) of cells and/or by matrix material, protein, etc. “Encapsulated cell technology” includes encapsulation utilizing microparticles, microspheres, microbeads, microcarriers, microcapsules, microgels, hydrogels, nanocarriers and nanoparticles, and combinations thereof.

This invention prevents risk to the patient by including in some embodiments specific encapsulation methods to prevent the direct contact of hybrid cells to patient's immune system.

In this invention, the use of encapsulated cell technology is also extended to include the encapsulated maturation of the hybrid cells during their development in vitro. By in vitro encapsulation methods, any herein claimed hybrid cell(s) of more uniform properties can be derived allowing more consistency in cellular morphology, stability characteristic and functionality.

Encapsulated cell technology in this respect offers co-culturing of cells within the encapsulation matrix resulting in the confinement of the cells and/or limiting their spatial expansion.

Immediate encapsulation of a desired population(s) of mixed hybrid cell and/or of unfused cells allows for a synergistic development. Synergistic properties include the sum of all benefits that can be derived from having cells grow in proximity to each other. Thus, by virtue of this process, constraints in space would prevent hybrids or, for that matter, any of the surrounding cancer cells from growing uncontrollably. By definition a cancerous cell is one that grows uncontrollably. Intact spatially constrained encapsulation does not allow the spread of cells beyond the confines of the encapsulation matrix and are therefore not cancerous.

The constraints in space would also limit the reductive division of the polyploid cells allowing their increased stability and/or functionally due to enhanced energy and mRNA content.

Confinement allows reduces apoptosis and cell necrosis, plus provides for an improved microenvironment due to paracrine signaling.

Finally, it must be pointed out that encapsulation allows for selection and de-selection of specific population of cells for achieving optimal homing characteristics.

In one embodiment: Hybrid cells can be added to extracellular matrix (ECM) and are thereafter immediately encapsulated and monitored for cell migration (homing) by phase contrast microscopy.

ECM can be manipulated to include ligands for homing of hybrid cell receptors and growth factors.

In a preferred embodiment: Encapsulation is achieved by the method of Cellesia et al, (2004) using a synthetic substitute for alginate, incorporated herein by reference.

It is understood that multilayering of encapsulation can be also performed (Schneider et al, 2001, incorporated herein by reference) and would be preferred wherein a single-layered encapsulation would have safety issues of leakage, breakage or antigenicity of a single-layered encapsulation. Moreover, multilayering would not affect the transport of gas exchange, nutrients and waste products to cause cellular necrosis.

The invention herein pertaining to encapsulation is a step of “cellular conditioning” before or after the fusion step which differs from the closest art (all incorporated herein by reference) in that in the closest art;
(1) cells are deposited on acellular particulate tissue matrix which does not encapsulate the cells within a restricted environment (Griffey et al, U.S. Pat. Appl. No. 20050159822);
(2) (alginate has been ionically cross-linked to form a hydrogel matrix and used for encapsulation of already established hybridoma cells, (Lim U.S. Pat. No. 4,352,883; Kihm et al, U.S. Pat. Appl. No. 20050058631);
(3) general instances of encapsulation of already established stem cells are used to evade the immune system and for convenient implantation and retrieval (European Patent Publication No. 301,777 or U.S. Pat. Nos. 4,353,888; 4,744,933; 4,749,620; 4,814,274; 5,084,350; 5,089,272; 5,578,442; 5,639,275; and 5,676,943, Morrison et al, U.S. Pat. Appl. No. 20040110288, Rudnicki and Seale, U.S. Pat. Appl. No. 20050042637; Tresco et al., 1992, ASAIO J. 38, 17-23; Aebischer et al., 1996, Hum. Gene Ther. 7, 851-860; Akzo Nobel Faser A G, Wuppertal, Germany; D glon et al, 1996, Hum. Gene Ther. 7, 2135-2146); and
(4) general instances of encapsulation of established cells are used for treatment of diabetes (Lim et al., Science, 210:908 (1980); O'Shea et al., Biochim. Biophys. Acta., 840:133 (1984); Sugamori et al., Trans. Am. Soc. Artif. Intern. Organs, 35:791 (1989); Lacey et al., 1991, Science, 254:1782; Levesque et al., Endocrinology, 130:644 (1992); and Lim et al., Transplantation, 53:1180 (1992); Aebischer et al., U.S. Pat. No. 4,892,538; Aebischer et al., U.S. Pat. No. 5,106,627; Hoffman et al., Expt. Neurobiol., 110:3944 (1990); Jaeger et al., Prog. Brain Res., 82:4146 (1990); and Aebischer et al., J. Biomech. Eng., 113:178-83 (1991)), or can be co-extruded with a polymer which acts to form a polymeric coat about the .beta. islet cells (Lim, U.S. Pat. No. 4,391,909; Sefton, U.S. Pat. No. 4,353,888; Sugamori et al., Trans. Am. Artif. Intern. Organs, 35:791-99 (1989); Sefton et al., Biotechnol. Bioeng., 29:113543 (1987); Aebischer et al., Biomaterials, 12:50-55 (1991); and Gryseels et al, U.S. Pat. Appl. No. 20040096967).

This invention further provides use of any herein claimed hybrid cell(s) or resultant culture or graft thereof for any other applications not limited to drug screening, drug discovery and cosmetic surgery. Included in the scope of this invention are cross species recipients within the same biological classification. It is understood that any herein claimed hybrid cell(s) or resultant culture or graft thereof, may be combined in the same encapsulation, or separate encapsulations combined within the same multilayered encapsulation, or multiple encapsulations thereof combined in and/or on an animal.

For placement of the invention herein within or on a mammal, or aves, various therapeutic delivery systems are known and can be used to administer the invention, e.g., alginate, gelatin, albumin, liposomes, microparticles, microcapsules, microspheres, receptor-mediated endocytosis (see, e.g., Wu and Wu, 1987, J. Biol. Chem. 262:4429-4432, incorporated herein by reference), encapsulation in a synthetic substitute of alginate including polyacrylates with thermal gelation and chemical cross-linking, etc. Methods of administering a prophylactic or therapeutic amount of the invention include, but are not limited to, parenteral administration (e.g., intradermal, intramuscular, intracoronary, intraperitoneal, intravenous and subcutaneous), epidural, and mucosal (e.g., intranasal, inhaled, and oral routes). The invention may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the invention into the central nervous system by any suitable route, including intraventricular and intrathecal injection; intraventricular injection may be facilitated by an intraventricular catheter, for example, attached to a reservoir, such as an Ommaya reservoir. Similarly, various external devises can be used to house the invention herein on a mammal, or avian, and injection of the invention herein and/or a bio-pharmaceutical product(s) from the invention may occur.

For the placement of the invention herein within or on a mammal, or aves, the term “cellular composition” may apply and refers to a preparation of cells, which preparation may include, in addition to the cells, non-cellular components such as cell culture media, e.g., proteins, amino acids, nucleic acids, nucleotides, co-enzyme, anti-oxidants, metals and the like. Furthermore, the cellular composition can have components which do not affect the growth or viability of the cellular component, but which are used to provide the cells in a particular format, e.g., as polymeric matrix—for encapsulation or a pharmaceutical preparation.

Examples of matrices that may be used for the formation of donor tissues or organs include collagen matrices, carbon fibers, polyvinyl alcohol sponges, acrylateamide sponges, fibrin-thrombin gels, hyaluronic acid-based polymers, and synthetic polymer matrices containing polyanhydride, polyorthoester, polyglycolic acid, or a combination thereof (see, for example, U.S. Pat. Nos. 4,642,120; 4,846,835; 5,041,138; and 5,786,217 all incorporated herein by reference).

In the inventions herein regarding cellular fusions it is understood that cellular hybrids of the simple 4N karyotype product of a 2N LCSO cell with a 2N SC cell may be produced, and can be represented with the equation;
Hybrid Karyotypes=LCSO .sub.XN SC .sub.YN.,
where “X” and “Y” are karyotype integers that are initially multiples of two. Subsequently karyotype noninteger values occur being from loss of a specific chromosome(s)). Examples of alternate hybrid karyotypes are presented.
(1) A LCSO cell fuses with an SC cell and then fuses with another SC cell, with resultant Hybrid Karyotypes=LCSO .sub.2N SC .sub.4N.
(2) A SC cell fuses with another SC cell and then fuses with a LCSO cell, with resultant Hybrid Karyotypes=LCSO .sub.2N SC .sub.4N.
(3) A LCSO cell fuses with another LCSO cell, then fuses with an SC cell, with resultant Hybrid Karyotypes=LCSO .sub.4N SC .sub.2N.
(4) A LCSO cell fuses with an SC cell and then fuses with another SC cell, and then undergoes chromosomal loss in the range of 0.5N to 3.0N during “conditioning”, with resultant Hybrid Karyotypes=a range from LCSO .sub.2N SC .sub.1 N. to LCSO .sub.2N SC .sub.3.5N.
(5) It is understood that the above hybrid karyotypes can occur for all hybrid cells in this invention herein.

### BRIEF DESCRIPTION OF THE DRAWINGS

Not Applicable.

### DETAILED DESCRIPTION OF THE INVENTION

All references, patents and patent publications that are recited within the “Detailed Description of the Invention” are incorporated in their entirety herein by reference.

Porcine Pancreatic Cells. Methods for obtaining suitable LCSO cells (or NL-LCSO cells) from an animal source are well known in the art. For example (as described in U.S. Pat. No. 4,195,125), for LCSO cells being an islet cell from an animal such as porcine, pancreatic tissue was removed from pigs and kept for 20 to 40 minutes in Earle's salt solution (ESL) supplemented with 200 ug penicillin and 200 ug streptomycin/ml, at 4.degree.C. The pancreas tissue, freed from connective tissue and fat, was cut into pieces 1.times.1 mm in size with scissors and washed repeatedly prior to being digested with collagenase. For the digestion, a 1 g portion of the tissue was incubated for 8 to 10 minutes at 30.degree.C. to 37.degree.C. with 1 ml collagenase (17449 SERVA, Heidelberg, 0.6-0.8 U/mg) per ml ESL with shaking. To the resultant tissue suspension was added the double quantity of Dulbecco's MEM-modification (DMEM) supplemented with 10% calf serum and the supernatant was decanted after sedimentation of the tissue portions. This procedure was repeated using 4 to 6 portions of fresh collagenase solution. The supernatants of the first and second enzyme treatments were discarded. The following collagenase fractions contained islets, endocrine cell groups and single cells. These supernatants kept at room temperature were centrifuged after the last treatment, absorbed in a little DMEM and drawn up and syringed through a 20 G 11/2 cannule until all islets and endocrine cell groups had disintegrated into single cells (about 5 to 10 ml). Then the cell suspension was centrifuged and washed 3 to 4 times with DMEM supplemented with 10% calf serum and antibiotics. The cell suspension consisting of about 80% of B-cells, the remainder being A-, C- and D-cells, was taken up in 10 ml DMEM supplemented with 10% fetal calf serum and antibiotics at a concentration of about 5.times.10.sup.5 cells/ml, seeded out in 10 cm Petri dishes (Corning, Wiesbaden) and incubated for 24 hours at 37.degree.C. in a 5% carbon dioxide atmosphere.

Human Islet Cells. Significant improvement have occurred for human islet isolation suitable for transplantation (Matsumoto et al, 2004a, 2004b). For pancreas procurement and preservation, the pancreata were immediately placed in chilled UW solution (University of Wisconsin, ViaSpan, DuPont Pharma) and transported to the laboratory. Upon arrival of the pancreas, islet isolation began immediately by the two-layer method (TLM, Matsumoto et al, 2002) in accordance to the Edmonton protocol (Shapiro et al, 2000). Before transferring the organ into the TLM, the spleen and excess fat tissue attached to the pancreas were carefully dissected away. The pancreas was transferred to a 1 L Nalgene container containing a solution of PFC (perfluorodecalin C10F18, F2 Chemicals Ltd., Preston, Lancashire, UK) and overlaid with UW. The pancreas was allowed to float between the two solutions while 100% oxygen was continuously bubbled into the PFC at a flow rate of 50 to 100 ml/min. The whole system was kept in a refrigerator and maintained at 2 to 8.degree.C. The pancreata were processed within 12 hours of placement into UW solution. The two-layer method was clinically sterile by using sterilized disposable suction chambers (Baxter Healthcare Corp., IL) for our two-layer chambers; sterilized tubes for oxygen inlet and outlet; sterilized 0.2-micrometer air filters (Gelman Science, MI). At the end of the preservation time we collected pancreas preservation fluid to assess it for bacterial contamination (Matsumoto et al, 2000).

The pancreas was decontaminated, rinsed, and partially bisected to gain access to the main pancreatic duct. Eighteen-gauge needles were inserted into both the head and tail of the main pancreatic ducts. A cold solution of enzyme consisting of Liberase-HI, (Roche Molecular Biochemicals, Indianapolis, Ind.) 24 mM HEPES buffer (Mediatech, Inc, Herndon, Va.) and 3.5 mM calcium dichloride (Sigma Chemical Co., St. Louis, Mo.) was injected at 60 to 80 mm Hg for the first 5 minutes and 160 to 180 mm Hg for the next 5 minutes (Lakey et al, 1999; Shapiro et al, 2000).

The distended pancreas was cut into 7 to 9 pieces and transferred to a Ricordi chamber (Ricordi et al, 1988). The pancreas was digested by recirculating the enzyme solution through the Ricordi chamber at 37.degree.C. When the majority of islets were free from exocrine tissue, the digestate was diluted with Media 199 (GibcoBRL, Grand Island, N.Y.) totaling approximately 9 L. Diluted digestate was collected into 225 ml conical tubes prefilled with 15 ml 25% human albumin (Bayer Corporation, Elkhart, Ind.) and kept cold on ice. The digestate was washed with cold Media 199 supplemented with 28 ml/L 25% human albumin.

Islets were purified using a continuous density gradient of high osmolality Ficoll (Pharmacia, Uppsala, Sweden) in an apheresis system (COBE 2991 cell processor, Gambro Laboratories, Denver, Colo.) according to the Edmonton protocol (Ricordi et al, 1989; Brandhorst et al, 1998; Shapiro et al, 2000; Ryan et al, 2001). The digestate was aliquoted in to 50-ml Falcon tubes and centrifuged at 1000 rpm for 2 minutes. The supernate was discarded and the pellet suspended in 15 ml of a solution containing 80:20 Lymphoprep:HBSS (with 2% human albumin). Then 10 ml of HBSS was layered over the Lymphoprep-HBSS medium. The tubes were centrifuged at 1800 rpm for 5 minutes at 4.degree.C. Islets recovered at the interface between the two layers were sedimented at 1800 rpm (2 minutes/4.degree.C.). The supernate was discarded and the pelleted islets aliquoted into 75-cm.sup.2 suspension flasks with M199 culture medium supplemented with penicillin (100 U/ml), streptomycin (100 ug/ml), gentamicin (50 ug/ml), and amphotericin B (0.25 ug/ml).

Only pure fractions (approximately more than 50% purity) were collected. For islet culturing and culture recovery, purified islets were cultured in CMRL 1066 culture media (Mediatech, Inc. Herndon, Va.) with 10% fetal bovine serum (FBS, Tissue Culture Biologicals, Tulare, Calif.) at 37.degree.C. in humidified air (5% CO.sub.2) for 18 to 24 hours.

The isolation results that yielded more than 100,000 IE (islet equivalents) postpurification and a stimulation index of 2.0 were considered successful isolations (Lakey et al, 1996). Samples were collected in duplicate for the quantification of the islets, expressed in terms of islet equivalents, the standard unit for reporting variations in the volume of islets, with the use of a standard islet diameter of 150 um. Islet recovery following purification was assessed in duplicate by counts of dithizone-stained aliquots of the final suspension of tissue (Ricordi et al, 1990). Purity of the preparation was assessed subjectively by comparing the relative quantity of dithizone-stained tissue to unstained exocrine tissue. In brief for the stimulation index, the islets were incubated for 24 hours at 37.degree.C. in CMRL 1066 medium with 10 percent fetal-calf serum and 25 mmol HEPES buffer. A known number of duplicate aliquots of islets were incubated in a low concentration of glucose (50 mg per deciliter [2.8 mmol per liter]) and a high concentration of glucose (360 mg per deciliter [20 mmol per liter]) for two hours, and the amount of insulin generated in response to the high-glucose challenge was divided by the amount generated by the low-glucose challenge to yield the mean insulin-release stimulation index (Korbutt et al, 1996). Islet morphology was assessed by light and electron microscopy (Marchetti et al, 2002).

Small Animal Islet Isolation. Islet isolation from small animals has been automated (Gurol et al, 2004). Pancreata of Wistar albino rats (220 to 240 g), were inflated with 5 mg Collagenase P (Boehringer Mannheim, Mannheim, Germany) diluted in 10 ml Hank's solution. Pancreatic tissue was disrupted mechanically and placed into the isolation chamber with an additional 10 mg of Collagenase P (Roche Diagnostics). A system is displayed and described with two chambers: one for isolation of islets and one for recirculation and collection. The volume of the isolation chamber including the circulation tubes in the peristaltic pump was 70 ml. The viability of the obtained islets examined by Trypan blue exclusion.

Human HPCs Isolation. Human HPCs were either aliquots of leukapheresis products obtained from healthy individuals donating G-CSF-mobilized CD34+ cells for allogeneic transplantation or were remaining backup samples of cryopreserved leukapheresis products from patients after autologous stem cell transplantation. The light-density (<1.077 g/cm.sup.3) cells were isolated from the samples by centrifugation on Ficoll-Hypaque (Pharmacia, Uppsala, Sweden) and then cryopreserved in fetal calf serum (FCS; GibcoBRL, Invitrogen GmbH, Paisley, U.K.) with 10% DMSO (Sigma, St. Louis, Mo.) at −180.degree.C. Cells were subsequently thawed, and cell populations expressing mature erythroid, granulopoietic, megakaryopoietic, and lymphoid markers were removed using a StemSep.™ column (Stem Cell Technologies, Vancouver, Canada), resulting in a lineage-depleted (lin-) cell fraction enriched for CD34+ cells. For some experiments, a positive selection of CD34+ cells using an Isolex.™ 300i column (Nexell Therapeutics, Irvine, Calif.) was performed, thus yielding two different populations of human HPCs (Hart et al, 2004).

Murine BMC Isolation. Murine BMCs were flushed from the femoral and tibial bones of the BALB/c mice and then suspended in RPMI. The BMCs were then filtered through a 70-mm nylon mesh (Becton Dickinson Labware; Franklin Lakes, N.J.), washed, and adjusted to 1.5.times. 10.sup.9 cells/ml in RPMI. The BMCs, thus prepared, are suitable for BMC transplant by direct injection into the bone cavity (intra-BM injection (IBM)) (Nakamura et al, 2004).

Rat Embryonic Neural Progenitor Cells Isolation. Timed pregnant Sprague-Dawley rats at E14.5 were purchased from Harlan Sprague-Dawley (Indianapolis, Ind.). E14.5 Sprague-Dawley rat embryos were used to isolate neural progenitor cells as previously described (Kalyani et al, 1997). Briefly, the rat embryos were removed and placed in a Petri dish containing ice-cold phosphate-buffered saline (PBS; Invitrogen). The trunk segments of the embryos (last 10 somites) were dissected, rinsed, and then transferred to fresh cold PBS. All isolated embryonic neural tubes and tissues derived from adult rats were stored in an RNAlater solution (Ambion; Austin, Tex.) at 4.degree.C. for late RNA isolation (Luo et al, 2003).

Human Fetal Neural Progenitor Cells Isolation and Propagation into Neurospheres. Human fetal brains (FBr) were obtained from Advance Bioscience Resources, in accordance with all state and federal guidelines. FBr tissue was minced and then dissociated enzymatically in a solution containing 0.1% collagenase (Roche) and 0.1% hyaluronidase (Sigma, St. Louis, Mo.) at 37.degree.C. for 1 hour. The cells were further treated with 0.05% trypsin-0.53 mM EDTA (Gibco, Grand Island, N.Y.) for 10-15 minutes to obtain a single-cell suspension for mAb staining and cell sorting for CNS-SC isolation. Typically 1-10.times.10.sup.8 cells were obtained from each FBr tissue of 16-20 gestational weeks. Cells were resuspended in Hank's balanced salt solution (HBSS) buffer containing 0.1% human serum albumin (Gilfol) and 10 mM HEPES (Gibco) (Tamaki et al, 2002).

The staining and sorting of CNS-SC from FBr were described previously (Uchida et al., 2000). Briefly, the dissociated FBr cells were incubated with mAb against CD34 fluorescein isothiocyanate (FITC; BD Bioscience), CD45-FITC (Caltag, South San Francisco, Calif.), CD24-APC (8G1; Stem-Cells, Inc., Palo Alto, Calif.), CD133/1 and CD133/2-PE (Miltenyi Biotech), or CD34 FITC (BD Bioscience), CD45-FITC (Caltag), CD24-PE (8G1; Stem Cells, Inc.), and CD133/1 and CD133/2-biotin (Miltenyi Biotech), followed by streptavidin-APC (Caltag) for 20-60 minutes. Stained cells were washed and resuspended in HBSS containing 0.1% human serum albumin, 10 mM HEPES (Gibco), and 0.5 ug/ml propidium iodine (PI) and sorted with a dual-laser Vantage SE (BDIS) (Tamaki et al, 2002). Long-term human neurosphere cultures from FBr have been described previously (Carpenter et al., 1999; Uchida et al., 2000). Briefly, the fluorescence-activated cell sorted (FACS) cells were cells were seeded at approximately 100,000 cells/ml and cultured in human neurosphere culture media consisting of X-vivo 15 medium (BioWhittaker), N-2 supplement (Gibco), and 0.2 mg/ml heparin supplemented with basic fibroblastic growth factor (b-FGF, 20 ng/ml), epidermal growth factor (EGF, 20 ng/ml), and leukemia inhibitory factor (LIF, 10 ng/ml). Cultures were fed weekly and passaged at 2-3 weeks. The neurospheres were passaged by harvesting the cells and then enzymatically dissociating the spheres into a single-cell suspension (Lefkovits and Waidmann 1999) or reseeded at approximately 75,000-100,000 cells/ml in the presence of collagenase [0.5 mg/ml in phosphate-buffered saline (PBS) containing 0.1% HSC] for 5-10 minutes (Tamaki et al, 2002).

Murine Adult Pancreatic Stem Cells. The mice were 6-week-old male GFP animals constitutively expressing GFP in all cells (Jackson) and wild-type BalbC animals (Charles River). Islets were isolated by collagenase digestion of the pancreas and Ficoll density gradient centrifugation. After centrifugation islets were handpicked for further purification. Ductal tissue was similarly handpicked to ensure purity (Seaberg et al, 2004).

Isolated islets and ductal tissue were then incubated with trypsin (Sigma) at 37.degree.C. and triturated with a small-borehole siliconized pipette into a single cell suspension. Viable cells were counted using Trypan Blue (Sigma) exclusion and plated at 20 cells/ul or less in defined serum-free medium (SFM) (Troupepe et al, 1999). SFM was composed of a 1:1 mixture of Dulbecco's modified Eagle's medium (DMEM; GIBCO) and F-12 nutrient (GIBCO) including 0.6% glucose (Sigma), 2 mM glutamine (GIBCO), 3 mM sodium bicarbonate (Sigma), and 5 mM HEPES buffer (Sigma) with a defined hormone and salt mixture (Sigma) that included insulin (25 mg/ml), transferrin (100 mg/ml), progesterone (20 nM), putrescine (60 mM), and selenium chloride (30 nM). Defined SFM additionally contained 1×B27 (Gibco-BRL), 10 ng/ml FGF2 (Sigma), 2 ug ml/ml heparin (Sigma) and 20 ng/ml EGF (Sigma) for 7-14 day in vitro. For some experiments, the following growth factors were added: 100 pM hepatocyte growth factor (Sigma), 10 ng/ml keratinocyte growth factor (Calbiochem), 10 ng/ml insulin-like growth factor-1 (Upstate Biotech), 2 nmol/LActivin-A (Sigma), 10 mM nicotinamide (Sigma) and 10 nM exendin-4 (Sigma) (Seaberg et al, 2004).

For clonal analysis, primary cells were diluted to a density of 0.05 cells/ul and plated in Nuncion 96-well plates (Nalge Nunc International). Each well was scored after plating for the presence of a single cell. Only wells that contained single cells at the outset of the culture period were subsequently assayed for colony formation (Seaberg et al, 2004).

For differentiation, whole individual pancreas colonies were removed from the aforementioned mitogen-containing media and transferred to wells coated with Matrigel basement membrane matrix (15.1 mg/ml stock diluted 1:25 in SFM, Becton-Dickinson) in SFM containing 1% FBS without dissociation. As the colony differentiates, cells migrate out of the spherical colony to form a flat monolayer. To ensure accurate assay of the progeny from single pancreatic precursors, each well contained only a single clonal pancreas colony (Seaberg et al, 2004).

Human Umbilical Cord Mesenchymal Stem Cell-Like Cells isolation and Propagation. Medium 199 with Earie's salts, Dulbecco's modified Eagle's medium with low glucose (DMEM-LG), Dulbecco's phosphate-buffered saline (PBS), Earie's balanced salt solution (EBSS), penicillin-streptomycin, L-glutamine, sodium pyruvate, and trypsin-EDTA were obtained from GIBCO Invitrogen Corp. (Paisley, Scotland, UK). Fetal bovine serum ([FBS] preselected for the growth of human mesenchymal cells) was obtained from StemCell Technologies (Vancouver, Canada). Collagenase type IV, bovine serum albumin (BSA), and Triton X-100 were acquired from Sigma Chemical Co. (St. Louis, Mo.). Cell culture plastic was from Corning Inc. (Corning, N.Y.) and Sigma-Aldrich (Romanov et al, 2003).

Umbilical cords (n=26; gestational ages, 39-40 weeks) were collected and processed within 6-12 hours after normal deliveries. The cord vein was canulated on both sides and washed out with EBSS. The vessel was filled with 0.1% collagenase in Medium 199 supplemented with antibiotics and incubated at 37.degree.C. for 15 minutes. The vein was then washed with EBSS and, after gentle “massage” of the cord, the suspension of endothelial and subendothelial cells was collected. The cells were centrifuged for 10 minutes at 600 g and resuspended in DMEM-LG supplemented with 20 mM HEPES, 100 U/ml penicillin, 100 ug/ml streptomycin, 2 mM L-glutamine, 1 mM sodium pyruvate, and 10% FBS. After counting, cell suspension was seeded in noncoated 75-cm.sup.2 culture flasks with a density of approximately 10.sup.3 cells/cm.sup.2. Cultures were maintained at 37.degree.C. in a humidified atmosphere containing 5% CO.sup.2, with a change of culture medium every other day. Approximately 2 weeks later, when well-developed colonies of fibroblast-like cells appeared, cultures were washed with EBSS, harvested with 0.05% trypsin-0.02% EDTA, and passaged (without dilution) into a new flask for further expansion (Romanov et al, 2003).

hES Cell Line Isolation. Fresh or frozen cleavage stage human embryos, produced by in vitro fertilization (IVF) for clinical purposes, were donated by individuals after informed consent and after institutional review board approval. Embryos were cultured to the blastocyst stage, 14 inner cell masses were isolated, and five ES cell lines originating from five separate embryos were derived, essentially as described for nonhuman primate ES cells (Thomson et al, 1995 and 1998).

Thirty-six fresh or frozen-thawed donated human embryos produced by IVF were cultured to the blastocyst stage in G1.2 and G2.2 medium (Gardner et al, 1998). Fourteen blastocysts were selected for ES cell isolation, as described for rhesus monkey ES cells (Thomson et al, 1995). The inner cell masses were isolated by immunosurgery (Solter and Knowles, 1975), with a rabbit antiserum to BeWO cells. Six days after ovulation, an azonal blastocyst was recovered by a nonsurgical uterine flush technique from a 15-year-old rhesus monkey (Seshagiri et al, 1993). The trophectoderm was removed by immunosurgery (Solter and Knowles, 1975), using a rabbit anti-rhesus spleen cell antiserum followed by exposure to guinea pig complement. Rabbit anti-mouse-spleen serum was produced in a New Zealand White rabbit, which was bled 10 days after three intravenous injections of 4.times.10.sup.8 ICR mouse spleen cells. Serum was heated at 56.degree.C. for 30 minutes before use to inactivate rabbit complement. Fresh guinea pig serum was used as the source of complement at a final dilution of approximately 1:16 (Thomson et al, 1995).

The intact inner cell mass (ICM) was separated from lysed trophectoderm cells. The intact inner cell masses were plated on irradiated (35 grays gamma irradiation) mouse embryonic fibroblasts. Culture medium consisted of 80% Dulbecco's modified Eagle's medium (no pyruvate, high glucose formulation; Gibco-BRL) supplemented with 20% fetal bovine serum (Hyclone), 1 mM glutamine, 0.1 mM 2-mercaptoethanol (Sigma), and 1% nonessential amino acid stock (Gibco-BRL). After 9 to 15 days, inner cell mass-derived outgrowths were dissociated into clumps either by exposure to Ca.sup.2+/Mg.sup.2+-free phosphate-buffered saline with 1 mM EDTA (cell line H1), by exposure to dispase (10 mg/ml; Sigma; cell line H7), or by mechanical dissociation with a micropipette (cell lines H9, H13, and H14) and replated on irradiated mouse embryonic fibroblasts in fresh medium. Individual colonies with a uniform undifferentiated morphology were individually selected by micropipette, mechanically dissociated into clumps, and replated. Once established and expanded, cultures were passaged by exposure to type IV collagenase (1 mg/ml; Gibco-BRL) or by selection of individual colonies by micropipette. Clump sizes of about 50 to 100 cells were optimal. Cell lines were initially karyotyped at passages 2 to 7 (Thomson et al, 1995).

The resulting cells had a high ratio of nucleus to cytoplasm, prominent nucleoli, and a colony morphology similar to that of rhesus monkey ES cells. Three cell lines (H1, H13, and H14) had a normal XY karyotype, and two cell lines (H7 and H9) had a normal XX karyotype. Each of the cell lines was successfully cryopreserved and thawed. Four of the cell lines were cryopreserved after 5 to 6 months of continuous undifferentiated proliferation. The other cell line, H9, retained a normal XX karyotype after 6 months of culture and has now been passaged continuously for more than 8 months (32 passages). A period of replicative crisis was not observed for any of the cell lines (Thomson et al, 1998). For in vitro differentiation, cell line cells were plated at low density (5000 cells/cm.sup.2 of surface area) in the absence of fibroblasts on gelatin-treated four-well tissue culture plates (Nunc) in the same medium as that used for initial cell line isolation, but with 0-104 units of added human LIF per ml (GIBCO). The resulting differentiated cells were photographed 8 days after plating (Thomson et al, 1995).

hES Commercially Obtained and Propagated. hES cell lines BG01 and BG02 were obtained from BresaGen (Athens, Ga.) and cultured according to manufacturer instructions. ES cells were maintained on mitomycin-C-inactivated mouse embryonic fibroblast (MEF, from strain SVB, 1.times.10.sup.6 cells/35 mm dish) feeder cells in Dulbecco's-modified Eagle's medium/Ham's F12 (1:1) supplemented with 15% fetal bovine serum (FBS), 5% knockout serum replacement (KSR), 2 mM nonessential amino acids, 2 mM L-glutamine, 50 μg/ml Penn-Strep (all from Invitrogen; Carlsbad, Calif.), 0.1 mM 2-mercaptoethanol (Specialty Media; Phillipsburg, N.J.), and 4 ng/ml of basic fibroblast growth factor (bFGF; Sigma; St. Louis, Mo.). Cells were passaged by incubation in cell dissociation buffer or trypsin (Invitrogen), dissociated, and then seeded at about 20,000 cells/cm.sup.2. Under such culture conditions, the ES cells were passaged every 4-5 days. For freezing, cells were resuspended in medium containing 25% FBS, 65% hES medium, and 10% dimethylsulfoxide at 1.times.10.sup.6 cells/ml at approximately 1.degree.C. per minute, (Zeng et al, 2004).

hES Differentiation In Vitro. ES cell cultures were dissociated into small clumps by collagenase IV (Sigma) by incubating at 37.degree.C. for 5 minutes. The hES cell colonies were pelleted, resuspended in hES medium without bFGF (differentiation medium), and cultured in 6-well plates for 7 days with a medium change every second day. ES cell colonies grew in suspension as embryoid bodies (EBs), while remaining feeder cells adhered to the plate. The EBs were transferred into a new plate and were further cultured for 7 days before immunostaining, (Zeng et al, 2004).

hEG Isolation. The following example is taken from Turnpenny et al, 2003. With local research ethics committee approval and written informed consent, human fetuses at 7-9 weeks postconception were collected at termination of pregnancy. We initiated primary cultures of gonad cells from over 60 fetuses. Dissection was carried out using stereomicroscopy, and gonads were washed in Hanks' balanced salt solution (HBSS) (Sigma Chemical Co.; St. Louis, Mo.). Gonads were immersed in 0.01% EDTA for 10 minutes, washed in HBSS, then mechanically disaggregated and incubated in a cell dissociation mix, consisting of 0.25% collagenase, 20 U/ml DNase I (both from Sigma), 2% heat-inactivated newborn calf serum (Invitrogen Life Technologies Ltd; Paisley, UK), and 60 ug/ml CaCl.sub.2, in HBSS at 37.degree.C. for 1-2 hours, with repeated trituration. Cells were washed in HBSS and filtered through sterile gauze prior to plating (Turnpenny et al, 2003).

hEG Propagation. The following example is taken from Turnpenny et al, 2003. Mouse STO fibroblasts (American Type Culture Collection CRL-1503) were mitotically inactivated by exposure to 50 Gy of gamma-radiation and plated in Dulbecco's modified Eagle's medium (DMEM) with 10% fetal bovine serum (FBS), 100 U/ml penicillin, and 100 ug/ml streptomycin (all from Invitrogen). Dissociated primordial germ cells (PGCs) were plated onto this feeder layer in Knockout (KO)-DMEM (Invitrogen), containing either 15% KO serum replacement (KO-SR) (Invitrogen) or ES-cell-tested FBS (PAA Laboratories Ltd.; Pasching, Austria), 1 mM L-glutamine, 0.1 mM 2-mercaptoethanol (both from Sigma), 0.1 mM nonessential amino acids (Invitrogen), and antibiotics as above. To promote their survival, proliferation, and maintenance in the undifferentiated state, PGCs/EG cells were cultured in the presence of 10 uM forskolin (Sigma), 4 ng/ml human recombinant basic fibroblast growth factor (bFGF) (Cell Sciences; Norwood, Mass.), and 1,000 U/ml human recombinant leukemia inhibitory factor (LIF) (Chemicon International, Inc.; Temecula, Calif.). During the first 14 days, cultures were sacrificed or sampled for characterization (see below) or colonies were isolated by cloning cylinder and disaggregated with 0.25% trypsin/1 mM EDTA (Invitrogen) for 3-5 minutes at 37.degree.C. Cells were passaged onto fresh feeder layers, and samples were taken for additional characterization. Subsequently, selected cultures were replated onto one of several tissue culture surfaces: 0.1% gelatin, collagen, poly-L-lysine, or culture plastic. To promote differentiation, cells were either left to grow overconfluent or taken into suspension culture, accompanied by the withdrawal of LIF, bFGF, and forskolin from the culture medium. To encourage aggregation of cells in suspension, Ca.sup.2+ concentration was elevated to 4.5 mM (Vidricaire et al, 1994). Developing embryoid bodies were collected for individual culture in untreated round-bottom 96-well plates for periods ranging from 2 to 21 days. All cultures were maintained in 5% CO.sub.2/95% humidity at 37.degree.C. (Turnpenny et al, 2003).

Human Fetal Tissues; Preparation of Human Neural Tissues. Human fetal spinal cord (FSC) and brain tissues (FBr) were obtained from Advanced Bioscience Resources, in accordance with all state and federal guidelines. Minced FBr tissues were dissociated enzymatically in 0.1% collagenase (Roche Molecular Biochemicals) and 0.1% hyaluronidase (Sigma) at 37.degree.C. for 1 hour. Dissociated cells were further treated with 0.05% trypsin-0.53 mM EDTA (GIBCO) for 10-15 minutes to obtain a single cell suspension for cell sorting (1-10.times.10.sup.8 cells per tissue), (Uchida et al 2000).

Avian ES Isolation and Propagation.

The following avian examples (EXAMPLES 1-5) are taken from Petitte and Yang, U.S. Pat. No. 5,830,510.

### EXAMPLE 1

### Preparation of Feeder Cells

Gelatinizing culture dishes are prepared as follows. First, 0.1% gelatin is added to water to prepare a gelatin solution, which is then autoclaved. 4 ml of the gelatin solution is added to each plate for 6 cm plates, or 2 ml/well of gelatin solution is added to each well for 12-well plates. The plates or wells are incubated at 4.degree. C. for 30 minutes, and the gelatin aspirated prior to use.

STO feeder cells (American Type Culture Collection No. CRL 1503) are prepared by culturing STO cells to 80% confluency in DMEM with 10% FBS. The cells are then treated with mitomycin C at 10.mu.g/ml for 2-3 hours, after which they are rinsed three times with PBS. After rinse, the cells are trypsinized with a 0.25% trypsin/0.025% EDTA solution, the cells collected in DMEM with 10% FBS, and washed at 1,000 rpm for 5 min. After washing, cells are suspended in 5 ml of DMEM w.10% FBS and counted. The cells are then seeded onto gelatinized plates prepared as described above at a density of 1.times.10.sup.5 /cm.sup.2 and incubated overnight before use.

Primary Chicken Embryonic fibroblasts are prepared by harvesting fibroblasts from 10-day old chick embryos, subculturing the cells once, and then preparing the cells as feeder cells as listed for STO cells above.

### EXAMPLE 2

### Preparation of Conditioned Media

Buffalo Rat Liver (BRL) cell conditioned media is prepared by culturing BRL-3A cells (American Type Culture Collection No. CRL 1442) in DMEM w/10% FBS to confluency, then adding 13 ml of DMEM/10% FBS to each 75 cm.sup.2 flask. Media is collected from the flask every third day, with each flask being collected three to four times. Media is stored at −20.degree. C. For use, the media is filtered, adjusted to pH 7.5 with HCl, diluted to 80% BRL-CM with DMEM supplemented with 15% FBS, and the diluted conditioned media then supplemented with 0.1 mM .beta.-mercaptoethanol.

LMH (chicken liver cell) conditioned media is prepared by culturing LMH cells in the same manner as for BRL-3A cells above, and the conditioned media prepared in the same manner as BRL-conditioned media as given above.

### EXAMPLE 3

### Isolation of Unincubated Chick Embryo Cells

To isolate stages IX-XIV embryo cells, the surface of a fertilized chicken egg is sterilized with 70% ethanol, the egg opened, and the yolk separated from the albumen. The yolk is then placed in a petri dish with the blastoderm in the uppermost position. A filter paper ring is placed over the blastoderm and the yolk membrane cut around the periphery of the ring. The filter paper ring with the embryo is then transferred to PBS with the ventral side uppermost, excess yolk removed, the embryo teased from the yolk membrane, the embryo transferred to cold PBS and rinsed with PBS. PBS is then removed, trypsin added, and the embryo incubated for 10 min. at 4.degree. C. DMEM/10% FBS is added, the cells pelleted by centrifugation, the supernatant removed, and the cells resuspended in 80% BRL-CM. Embryo cells are then seeded onto the appropriate culture system.

### EXAMPLE 4

### Culturing of Avian Embryonic Stem Cells

Using the procedures given above several methods of culturing cell with an embryonic stem cell phenotype from unincubated chicken embryos were carried out. First, 10 whole embryos at stage X were isolated, dissociated, seeded onto chicken embryonic fibroblast feeder layers, and cultured with 80% BRL-CM. A significant amount of differentiation occurred, mainly cells of a fibroblast-like phenotype. Only a few clusters of cells remained relatively undifferentiated and contained large amounts of lipid. These cells grew slowly, if at all, and were lost by the second passage.

Second, 10 whole embryos at stage X were isolated, dissociated, seeded onto STO feeder layers, and cultured with 80% BRL-CM. Upon culture, the cells attached to the feeder layer and grew as small flattened colonies. In the first 3 passages, the cells lost all lipid droplets and exhibited a phenotype and growth characteristics similar to that observed for murine and porcine embryonic stem cells. Specifically, the cells contained a large nucleus with a prominent nucleolus and relatively little cytoplasm (see FIG. 1 and FIG. 2). The cells grew in nests with a generally uniform phenotype. Each nest remained a single cell thick as it grew, a characteristic shared with porcine, but not murine, embryonic stem cells. Unlike either murine or porcine cells, the nests of chicken embryonic stem cells exhibited the tendency to invade the feeder layer, pushing the STO feeder cells to the side or growing underneath the feeder layer. It was possible to culture these cells for 23 passages. In general, 10.sup.6 cells were seeded onto a STO feeder layer in a 6 cm dish and in 2-3 days 2.times.10.sup.6 to 5.times.10.sup.7 CES cells could be obtained.

On the fifth passage, a portion of the CES cells were transferred to BRL-CM media alone. Initially, the CES cells grew rapidly and formed large ES-like colonies. When passed onto new gelatinized plates in BRL-CM, the cells differentiated into fibroblast-like cells, accumulated lipid droplets and died with the nest passage. Likewise, at the tenth passage a portion of the CES cells grown on STO cells and in BRL-CM were seeded onto STO feeder cells alone. These cells also became fibroblast-like, and could not be maintained on STO feeder cells alone. These observation suggest that the culture of chicken embryonic stem cells requires both a feeder layer and conditioned media.

### EXAMPLE 5

### Initiating and Maintenance of Chicken Embryonic Stem (CES) Cells

Chicken embryonic stem cells are initiated by isolating stage IX-XIV unincubated chicken embryos, and the area pellucida used as the source of cells for culture. Cells are seeded onto mitotically inactivated STO feeder layers with 80% BRL-CM and DMEM supplemented with 15% FBS and 0.1 mM p-mercaptoethanol. After the initial seeding, the phenotype of the chicken cells is observed daily. Eventually, a portion of the cells begin to lose their lipid droplets and begin to invade the feeder layer while remaining a closely packed nest of cells generally one cell thick. During the first few passages, the entire culture is passed onto new STO feeder layers until several nests of stem cell-like cells appear.

Once an initial culture shows several nests of stem cells, the cultures are maintained by trypsinizing the culture and counting the number of chicken embryonic stem cells. About 0.3 to 1.times.10.sup.6 CES cells are seeded onto new STO feeder layers. The cultures are fed twice each day with BRL-CM and passed onto new feeder layers every 2-3 days, depending upon the density of the CES cells. Using this procedure, CES cells have been maintained for 23 passages (approximately two months). Data are given in Table 1 below.

TABLE 1
Yield of CES cells with each passage. Chick embryo cells
were seeded at 3.5 .times. 10.sup.5 cells/6 cm plate at day
0.1 .times. 10.sup.6 cells were seeded with each passage.

| Day of Culture | Passage Number | Total CES .times. 10.sup.6 |
|---|---|---|
| 23 | 9 | 3.0 |
| 25 | 10 | 3.5 |
| 27 | 11 | 4.0 |
| 29 | 12 | 2.0 |
| 31 | 13 | 1.5 |
| 34 | 14 | 3.0 |
| 37 | 15 | 1.3 |
| 38 | 16 | 2.0 |
| 42 | 17 | 2.5 |
| 44 | 18 | 3.0 |
| 49 | 19 | 1.0 |
| 51 | 20 | 1.6 |
| 53 | 21 | 0.7 |

Methods for obtaining LCSO and SC cells suitable as fusion partners are well documented in the literature. For example, a particular method of isolating adult stem cells of the present invention, comprises the steps of:
obtaining cells from a non-embryonic animal source and optionally filtering the cells through a 20 um filter;
slow freezing the cells in medium containing at least 7.5% (v/v) dimethyl sulfoxide until a final temperature of −80.degree.C. is reached; and
culturing the cells.

It is understood that the terms “LCSO cell” “NL-LCSO cell” “SC cell” and “ESG cell” includes those genetically engineered by most standard means in the art such as;
transfecting with a DNA construct comprising at least one of a marker gene or a gene of interest,
selecting for expression of the marker gene or gene of interest, and
culturing the resultant cells.

For preparation of bio-pharmaceutical product(s) (including gene product(s)) any in vitro technique which provides for the production of bio-pharmaceutical products by propagation of any herein claimed hybrid cell(s) or resultant culture, graft or cell line thereof may be utilized. Such techniques for generating any herein claimed hybrid cell(s) include, but are not limited to, cell fusions utilizing the various hybridoma and related techniques (by Sendai virus, Kohler & Milstein, 1975. Nature 256:495-497 and 1976); the trioma technique; the human B-cell hybridoma technique (Kozbor et al., 1983. Immunol. Today 4:72) and the EBV hybridoma technique (Cole et al., 1985. In: Monoclonal Antibodies and Cancer Therapy (Alan R. Liss, Inc., pp. 77-96)). Additionally, bio-pharmaceutical products may be produced in germ-free animals utilizing recently developed technology (PCT Publication US 90/02545). Bio-pharmaceutical products may be utilized in the practice of the present invention and may be produced by the method used for human hybridomas (Cote et al., 1983. Proc. Natl. Acad. Sci. USA 80:2026-2030) or by transforming said LCSOSC hybrid cells or said NL-LCSO-ESG hybrid cells with Epstein Barr Virus (EBV) in vitro (Cole et al., 1985. In: Monoclonal Antibodies and Cancer Therapy (Alan R. Liss, Inc., pp. 77-96).

Fusion methods for generating said LCSOSC hybrid cells or said NL-LCSO-ESG hybrid cells for in vitro or in vivo use include viral or chemical treatment methods (herein “conventional fusion techniques”), electrical, radiofrequency, laser or “natural fusion techniques”. Examples of conventional fusion techniques are (i) fusion using Sendai virus described by Kohler and Milstein (1975 and 1976) and White, J et al, J. Cell Biol. 89:674-679 (1981), and (ii) those using polyethylene glycol (PEG), such as 37% (v/v) PEG, by Gefter et al, (1977) and Davidson, R L, et al, Somatic Cell Genetics 2:271-280 (1976). The conventional fusion techniques have many shortcomings. Not only is the fusion yield often very poor, typically less than 0.01%, but the conventional fusion techniques may also cause severe side effects on the fused cells, thus greatly limiting their usefulness for many systems. The use of electrically induced fusion methods (electrofusion) is also appropriate (Goding pp. 71-74, 1986) and preferred in cases where conventional fusion techniques are not sufficient.

The PEG fusion method using porcine islet cells and murine stem cells is shown by example (U.S. Pat. No. 4,195,125 incorporated herein by reference) and is preferred for pancreatic or islet cells due to its historical success with these cells and the ease of use. 5.times.10.sup.6 pig cells and 1.times.10.sup.7 stem cells (a clonal derivative of the L-cell of the mouse) were detached from the Petri dishes by a trypsin treatment using 0.05% trypsin (LS—Labor Service, Munich) and 0.02% EDTA in a salt solution free of Ca.sup.2+ and Mg.sup.2+, mixed together and separated by centrifugation. The cell pellet was suspended by pipetting it 2 to 3 times using 0.5 ml PEG 6000 (SERVA, Heidelberg) as 50% solution in DMEM and was then incubated for 2 minutes at 37.degree.C. The agglutinated cell mixture was dispensed in 100 ml DMEM supplemented with 10% calf serum and 6% Ficoll placed in a cylinder of 2 cm diameter. The contents of the cylinder were allowed to stand for 3 hours at 37.degree.C. under sterile conditions, whereby the cells sedimented. The 10 ml bottom layer was taken up with a pipette, seeded out in DMEM in Petri dishes and incubated at 37.degree.C. in a 5% CO.sub.2 atmosphere. The fusion yield was about 5%. The stem cells, the pig cells, the heterocaryotic cells and the hybrid cells were left for growth for 3 days, then the used medium was withdrawn with a pipette. The cultures were overlaid with HAT-medium (Exptl. Cell Res. (1966) 41; 190) in which stem cells that had not fused stopped growing. After about 8 days the cultures were harvested by trypsinization and a small number of cells was again transferred to HAT-medium. In this process those cells that had not fused died as well. The hybrid clones had grown to full size in 3 to 4 weeks. Varying quantities of grown up hybrid cells were observed under the inverted phase contrast microscope. These clones comprising about 200 to 500 cells were placed in small steel cylinders 1.8 mm in diameter and were subjected to a trypsin treatment. The cell suspension was further cultivated in 6 cm Petri dishes. The hybrid cells were propagated in culture flasks (1200 ml) until the 35th passage was reached. After a trypsin treatment using a trypsin-EDTA solution (EDTA=ethylene diaminotetraacetic acid) a 150 ml portion of the cell suspension having a cell density of 1.times.10.sup.6 per ml of medium was seeded out in a 250 ml culture flask according to May (Zentralbl. Bakteriol. Abt. 1 Orig. (1964) 193, 306). The medium used was DMEM supplemented with 10% calf serum and 0.1 mg ZnSO.sub.4/liter. The stirring velocity was one rotation per second. The culture flask was placed in a water bath of 36.degree.C. A 5% CO.sub.2-air mixture was introduced into the culture by means of an aquarium pump by passing over a Millipore gas filter. A flow rate of 13 liter/hour was necessary in order to maintain a constant pH of 7.2. The number of cells had doubled every 2 to 3 days. Then approximately ⅓ of the cell suspension was withdrawn under sterile conditions and an identical quantity of medium was added. The cells that had been collected by centrifugation could be seeded out and continued to grow without difficulty. The insulin activity expressed in terms of immunologically measurable insulin in the supernatants of the collected cell suspensions (150 ml) depended on the quantity of glycose in the medium (300 mg percent) and was found to be about 15.mu.u U IMI after 6 hours of cultivation.

In this invention, it is understood that in some tissues, isolated individual cell types are not preferred for LCSO cells (or NL-LCSO cells). In treatment of diabetes the preferred LCSO cells are islet cells and next preferred pancreatic cells, over isolated beta cells. It is also understood that LCSO cells (or NL-LCSO cells) consisting of islet cells may be naturally contaminated with tissue resident adult stem cells (HSC, MSC or pancreatic stem cells) or transit amplifying cells (from HSC, MSC or pancreatic stem cells) and the preferred method is not to specifically remove these cells before fusion. Therefore in some cases, there may be fusion of an SC cell with adult stem cells or transit amplifying cells and that these may be incorporated into selected populations including into encapsulation.

It is understood in this invention, chemical fusion methods are not limited to PEG but can include any ingredient of those organic solvents or those dissolvable therein, including DMSO and DMF.

Alternative methods which induce cell fusion by electric fields have been developed. (Pohl, U.S. Pat. No. 4,476,004; Sowers, U.S. Pat. No. 4,622,302; Schoner, U.S. Pat. No. 4,578,167; Neumann, E. et al. Naturwissenschaften 67:414-415 (1980); Zimmerman, U. and Nienken, J., J. Membrane Biol. 67:165-182 (1982); Bates G. W., et al., Cell Fusion, Plenum Press pp. 367-395 (1987) all incorporated herein by reference). The basic principle of these methods of electrofusion by applying a pulsed high strength direct-current (DC) electric field across the cell is taken from U.S. Pat. No. 5,304,486 (incorporated herein by reference). This DC field is usually generated by briefly switching on a DC power source or by discharging a capacitor. The applied DC field has a strength of several kilovolts per centimeter. This external electric field induces a large cell membrane potential. When the membrane potential is of sufficient magnitude, a reversible breakdown of a small area of the cell membrane occurs. The breakdown results in the formation of physical pores at the surface of the cell. This process is called electroporation. Intracellular and extracellular material can exchange through the pore while it is open. After the DC field is removed, the pore will normally reseal quickly. When a pore is created between two closely adjacent cells a cytoplasmic bridge is formed via the pore. When the DC field is turned off the pore cannot reseal. Instead, the cytoplasmic bridge usually begins to enlarge, eventually causing the two cells to fuse.

Still, there are many limitations to electrofusion (U.S. Pat. No. 5,304,486). First, not all cell types can be fused with the same ease. In fact many cell types are extremely difficult to fuse with DC pulses. Second, there are many unknown factors which influence fusion yield. Fusion of certain cell types may be successful in one laboratory but not in others. The DC pulse(s) method is still more of an art than a well understood procedure. Third, it is very difficult to use the DC pulse(s) method to fuse cells of different sizes. This later problem occurs because the membrane potential induced by the external DC field is proportional to the diameter of the cell. Thus, the induced potential is larger for bigger cells. It is nearly impossible to chose a proper field strength of external field in order to fuse cells of two different sizes. When the external field is just sufficient to cause membrane breakdown in the larger cell, it is inadequate to induce a critical membrane potential in the smaller cell. Alternately, if the external field is elevated causing membrane breakdown in the small cell, the large potential induced in the larger cell will cause an irreversible membrane breakdown and cell death.

In this invention, radiofrequency pulsing induced cell fusion by Chang, D. C., (1989) is more preferred than electrofusion(s) in cases where electrofusion(s) are insufficient, overcoming the above electrofusion(s) problems. The high-power RF field produces an oscillating motion of the cell membrane through a process of electro-compression. Permeabilization of the cell membrane is caused by a combination of electrical breakdown and a localized sonication induced from the RF field. Thus, this oscillating electric field is more effective in breaking down the cell membrane than a DC field. Since this new method uses only physical means (i.e., RF electrical energy) to induce cell poration and cell fusion, it is free of biological or chemical contamination. The present invention produces results in seconds, provides much higher yields than conventional methods, and has minimal biological side effects. Thus, it is a clean, fast, efficient and safe method. The conventional methods of cell poration (including the DC field method) usually require a large number of cells (typically 5-10 million cells) to perform a gene transfection and, as a result, are unsuitable for use in human therapy in gene transfection. In contrast, the method of radiofrequency pulsing has been demonstrated to be able to transfect cells in small numbers with high efficiency, and will be highly useful for gene therapy.

Laser induced PEG cell fusion by Ohkohchi et al (2000) have also been described. Cell fusion is induced by using laser radiation after target cells are adhered to a surface with 5, 10, or 20% of PEG but without injuring the cells (best % PEG chosen by Trypan blue exclusion). A dish (tissue culture dish 3020, Falcon, Becton Dickinson Co.) with a hole at the bottom and covered with a thin glass was prepared. Under a microscope (Nikon Co., Tokyo, Japan) provided with a CCD camera, the target cell (SP2 cell used as a control) was first irradiated with 0.5 W at 1,064 nm by Nd-YAG laser (C-120, CVI-Laser Co., Albuquerque, N. Mex.) for trapping. The stage of the microscope was moved so that the trapped cell could come in contact with a second target cell. The second target cell was irradiated with another trapping laser beam (0.5 Wat 1,064 nm) and these two cells were fixed. The contact surface was irradiated with Nd-YAG laser (RCR-11, Quanta-ray, Spectra-Physics Co., Mountain View, Calif.) with a 1-3 mJ output at 355 nm 1-10 times. This procedure was monitored with the CCD camera (details described in Ohkohchi et al, 2000).

In this invention, fusion methods for generating hybrid cells comprise mixing LCSO cells with SC cells (or NL-LCSO cells with ESG cells) in a preferred 4:1 proportion, though the proportion may vary from about 20:1 to about 1:1, respectively, but are not limited to these ratios, in the presence of an agent or agents (viral or chemical (conventional fusion techniques), electrical, radiofrequency, or laser, or natural fusion techniques) that promote the fusion of cell membranes.

Conventional techniques of cell fusion usually produce viable hybrids at low frequencies, to as low as 1.times.10 sup.-6 to 1.times.10 sup.-8. However, even this does not pose a problem, as the viable, fused hybrids are differentiated from the parental, unfused cells (particularly the unfused SC cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azasenne. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The SC cells are defective in key enzymes of the salvage pathway, e.g., hypoxanthine phosphoribosyl transferase (HPRT), and they cannot survive. The LCSO cells (or NL-LCSO cells) can operate this pathway, but they have a limited life span in culture and generally die within about two weeks. Therefore, the only cells that can survive in the selective media are those hybrids formed from SC and LCSO cells (or NL-LCSO cells with ESG cells). This does not exclude using other cell selection means including FACS cell sorting or magnetic bead separation, which are preferred in cases where enrichment of a specific subpopulation(s) is required. In cases where the hybrid cells of this invention are to be placed on or within a species, the more preferred method is FACS cell sorting over HAT selection since genetic instability would not be introduced as a result of defective key enzymes of the salvage pathway, required for HAT selection.

The terms “condition”, “conditioned” and “conditioning” relating to cells are herein used to define the process by which the cells are exposed to a factor or to a multitude of factors that are generated as a result of cellular encapsulation whereby cells are held in proximity of each other in the presence or absence of coaxing factors.

In one preferred embodiment of this invention, encapsulated cell technology is used to “condition” LCSOSC hybrid cells (or NL-LCSO-ESG hybrid cells) in 3D before or after the cell fusion step, hereafter termed a step for “LCSOSC hybrid cell conditioning” abbreviated as LCSOSCC. LCSOSCC (or termed a step for “NL-LCSO-ESG hybrid cell conditioning” abbreviated as LCSO-ESGC). The preferred embodiment is given for LCSOSCC due to being more developmentally committed and therefore less likely to produce cancer, but it is understood that a parallel embodiment exists for LCSO-ESGC. LCSOSCC is the first part of a more gradual and controlled LCSOSC hybrid selection process directly after the fusion step. LCSOSCC contrasts the classical no growth media for unfused cell disposal directly after the fusion step. Instead of a selective no-growth agent, groups (populations) of cells from the mixture of cells after fusion will be encapsulated for about 1 to 5 weeks after fusion, during LCSOSCC. Thereafter, a selective growth agent step can be introduced or other selective means to exclude either unfused LCSO or SC cells or both. This LCSOSC hybrid cell conditioning step results in a higher proportion of more stable LCSOSC hybrid cells closer to the LCSO cell phenotype. This conditioning is unique since development, metabolic stabilization and genetic stabilization of LCSOSC hybrid cells will occur under the strong microenvironment influence of unfused quiescent LCSO cells, (“quiescent” meaning viable and at rest). LCSO cells become quiescent when contained in a fixed-size encapsulation and having no or little physical space to grow. This contrasts classical selection against the LCSO cell which allows LCSO cell growth and proliferation to achieve apoptosis or necrosis. The step for “LCSOSC hybrid cell conditioning” is the same step for “NL-LCSO-ESG hybrid cell conditioning” except different cells are used.

At the onset of LCSOSCC at encapsulation establishment, unique microenvironments will be set up. During LCSOSCC, there will be less metabolic pressure on the said LCSOSC hybrid cell, allowing it time to stabilize toward the LCSO cell phenotype. This is similar to the initial time of engraftment in vivo following transplantation where the stem cell or stem cell-like cell will be incorporated into the receiving tissue, “incorporation” meaning physically integrating with phenotype adoption. LCSOSCC also increases viability of said LCSOSC hybrid cells as relatedly observed by Tsuji, et al., (U.S. Pat. No. 4,916,072) for human hybridomas by pretreating the proliferative cells with a proliferation inhibitory agent before fusion. LCSOSCC in the preferred embodiment is performed in a 3D environment as a pellet of cells to mimic in vivo conditions. It has been well established that cell-cell interactions are distinctly different than in 3D versus 2D and therefore have great influence on phenotype. Bissell's group ground-breaking paper showed that antibodies against a cell-cell interaction surface receptor beta1-integrin completely changed the behavior of cancerous breast cells to a noncancerous phenotype in 3D but not 2D cultures (Weaver et al, 1997).

In another embodiment, LCSOSCC can be modified to enhance LCSOSC hybrid conditioning by additional substeps. Immediately prior to encapsulation a high concentration of or layers of an ECM protein, proteins or matrix, or any matrix preadsorbed by matrix metaloproteinase, cytokine, chemokine, or receptors thereof, apoptosis inhibitors, homing receptors or combinations thereof can be added, thereby being incorporated in the encapsulation. In a further embodiment substep, addition of space-filling matrix including any combination of ECM, glycogen amylopectin, gelatin, starch, mucins, glycosaminoglycans, polymerized albumin, or alginate-based or other hydrogels to the cells before a more rigid encapsulation will allow for limited growth within encapsulation as the inside space-filling matrix is broken down, self-dissolved or adsorbed by the cells. When the encapsulated space-filling matrix is unmodified calcium-alginate hydrogel with embedded microcrystals designed to dissolve on its own, space left by dissolved hydrogel can be filled in by growing cells, thus controlling growth within the fixed-sized encapsulation. A similar strategy can be obtained for space-filling matrix polymers containing protease or glycosidase sensitive bonds, with controlled levels of external proteases or glycosidases that can permeabilize the encapsulation, dissolve and secrete the space-filling matrix.

In a further embodiment, a “pro-selective encapsulation” substep (PSE) can be used at any time during LCSOSCC (or LCSO-ESGC in a further embodiment) and is considered part of the LCSOSCC (or LCSO-ESGC). A PSE substep is preferred for selecting either for or against various cell characteristics including homing of said LCSOSC hybrid cells and physically isolates them. A specific preferred embodiment including PSE at day 8 and 15 during LCSOSCC is given for LCSOSC hybrid cells with pro-selected characteristics of HSC-type homing. HSC-type of surface-bound homing ligands are required for this homing from the vessel to the bone marrow.

Prophetic PSE LCSOSCC example based on standard methodologies.

ECM can be made in vitro to mimic the in vivo sequence of homing steps required for the HSC-type homing LCSOSC hybrid cell. A small pellet of freshly fused human neutrophils (LCSO cells) and syngeneic human adult HSC(SC cells) (about 5000 to 100,000 cells) are first encapsulated in a reversible fully-synthetic substitute of alginate using (i) thermal gelation and (ii) chemical cross-linking (Cellesia et al, 2004). For thermal gelation the authors use a triblock copolymer of poly(ethylene glycol)-bl-poly(propylene glycol)-bl-poly (ethylene glycol) (Pluronics) that reversibly thermally gels in aqueous solution under physiological temperature and pH. The chemical cross-linking is then performed by a fully biocompatible proteolytically reversible cross-linking step. This step is a Michael-type addition occurring when a cold, slightly acidic medium (e.g. 5.degree.C., pH 6.8) is increased both in temperature and pH (37.degree.C., pH 7.4) and can be performed directly on the viable cells. This method is preferred being more biocompatible than the commercially available peptide hydrogel, PuraMatrix requiring cells at pH 3 for hydrogel formation. Cells are pelleted in a well onto a pre-polymerized layer, then sealed by a polymerization layer over the cells, forming the capsule. Encapsulation allows limited growth potential and cells are maintained viable for 1 week. Afterwards the population is taken out of the encapsulation (as thus termed “un-encapsulated”) by trypsinization and if necessary collagenized to obtain individual cells or small clusters of cells in suspension, pelleted, and allowed to propagate for 16 to 24 hours at a density minimizing cell-cell contact formation for acclimation and reestablishment of surface receptors. Thereafter the population is repelleted in a well onto a pre-polymerized layer, and a thin layer of synthetic ECM without fibronectin but containing E- and P-selectins is applied (simulating E- and P-selectins on the vessel endothelial wall) (Matrigel) followed by a thin layer of synthetic ECM with fibronectin (simulating ECM fibronectin in bone marrow stroma underlying the vessel endothelial wall), and re-encapsulated by polymerization layer over the ECM. During the next 6 to 48 hours incubation, the encapsulated cells are analyzed for cells that have migrated to the outer fibronectin-ECM layer (positive cells) by simple phase contrast microscopy for spatial location. Spatially positive cells are isolated upon proteolytic un-encapsulation and then divided at random into two populations to vary LCSOSCC length. The first, 8-day LCSOSCC population is clonally expanded into colonies and assayed for production of the restored gene product (for example ELISA of supernatant). The second, 15-day LCSOSCC population is pelleted and re-encapsulated, allowing the LCSOSC hybrid cell conditioning step to continue for 1 more week while restricting growth. This second population is again un-encapsulated and treated and assayed as in the first population for restored gene product. Restored gene product-positive colonies are first assayed for LCSO cell specific markers and separated from non-displaying cells to exclude unfused SC cells. Then selected cells are assayed cytologically by standard migration assay formats for proper homing characteristics as in the transwell migration assay (Liu et al, 2001). Finally additional cytological assays can be performed including energy charge in the cells for selection of the most metabolically viable clones (Ishii et al, 2004). During this clonal expansion period LCSO cells will be excluded. The LCSOSC hybrid cells passing all analyses will be grown into colonies if not already performed, then re-encapsulated and frozen (re-encapsulation increasing the viability upon thawing and is thus preferred, Rahman et al, 2004). Triblock copolymer encapsulation is not totally new as MacroMed commercially sells a triblock copolymer encapsulation material, ReGel for drug release with intratumoral injections (Zentner et al, 2001).

In this invention, a PSE substep as part of LCSOSCC is superior to the standard HAT selection method of said LCSOSC hybrid cells in that;
(1) the SC cell does not have to be made HPRT-negative for proper negative selection (although it may be incorporated),
(2) (necrotic or apoptotic cells are limited during the critical first weeks after fusion and therefore can not interfere with proper LCSOSC hybrid cell conditioning that occurs with viable cell-cell interactions or cytokine or chemokine release,
(3) time is saved by prescreening for homing populations versus individual clones for time consuming migration assay formats. Negative selection of SC cells can be performed by selection for LCSO cell specific surface markers by magnetic beads or FACS cell sorter by standard immuno-techniques.

At any time during LCSOSCC, it is understood that the following modifications may occur for optimization toward specific cell types used in fusion. During LCSOSCC the cells can be un-encapsulated, treated with ECM protein or proteins, chemokines, cytokines or apoptotic inhibitors, or combinations thereof and then re-encapsulated to continue LCSOSCC. Also, the cells can be un-encapsulated treated with a selective agent to reduce or eliminate a population of unfused cells, then re-encapsulated to continue LCSOSCC. Any one of these additional substeps can be performed more than once, be combined in any order, and can extend the LCSOSC hybrid conditioning step beyond the typical 5 weeks.

It is understood that at any time during LCSOSCC, monitoring for the restored bio-pharmaceutical product (including gene product) and cytologic cell surface marker or markers may occur between un-encapsulation and re-encapsulation. Alternately, monitoring can be performed by temporarily disrupting encapsulation long enough for monitoring reagents to penetrate the encapsulation, or rendering the monitoring agents small enough to diffuse through encapsulation or both.

Encapsulation, for LCSOSCC includes the art of “encapsulated cell technology” where a cell or cells are held within, or coated by, a polymer or gel layer (as classified by the USPTO) e.g., alginate, gelatin, albumin, or a semi-permeable polymer. Encapsulation of cells to escape immune surveillance while expressing a gene product to combat a diseased state are well known in the art. In choosing an encapsulation method, the microcapsule walls must have sufficient structural integrity and be sufficiently permeable for nutrients, and secretion and excretion products, to pass through, yet prevent the entry of molecules or cells of a host, for example, products of the host's immune response, which could destroy the encapsulated material. For example, the preferred encapsulation for insulin producing cells using alginate is reinforced alginate, and more preferably a fully-synthetic substitute of alginate using (i) thermal gelation and (ii) chemical cross-linking (Cellesia et al, 2004). “Encapsulated cell technology” also includes all medical device methods of sequestering cells from the environment including hollow fibers.

It is understood in this invention that the mixture of LCSO and SC cells (or NL-LCSO and ESG cells) can be encapsulated before fusion, and then fused, or fusion may occur in part before and in part after encapsulation. It is also understood that during encapsulation, cellular processes of the cell body may extend through the encapsulation material and may;
directly contact cells outside of the encapsulation material, or
form a synapse(s) with cells outside of the encapsulation material.

In this invention, culturing provides a population of any herein claimed hybrid cell(s) or resultant culture, graft or cell line thereof from which specific hybrid cells are selected for expansion to form hybrid cell lines. Typically, selection for cell lines is performed by, culturing the cells by single-clone dilution in microtiter plates, but may involve selection by small cell clusters or populations, followed by testing the individual clonal or cluster supernatants (after about two to five weeks) for the desired gene product. The assay should be sensitive, simple and rapid, such as radioimmunoassays, enzyme immunoassays, cytotoxicity assays, plaque assays, dot immunobinding assays, and the like.

The selected hybrid cells would then be serially diluted and cloned or clustered into individual gene product-producing cell lines, hybrid cell lines, which clones or clusters can then be propagated indefinitely to provide bio-pharmaceutical products (including gene products), cells or tissues. Cell clusters may have the distinct advantage of inherently maintaining or driving hybrid cells more toward the terminally differentiated phenotype of the LCSO cell (or NL-LCSO cell). The cell lines may be exploited for bio-pharmaceutical product production in two basic ways. A sample of the hybrid cells can be injected (often into the peritoneal cavity) into a histocompatible animal of the type that was used to provide the LCSO and SC cells (or NL-LCSO and ESG cells) for the original fusion. The injected animal develops tissues or cells secreting the specific gene product produced by the hybrid cells. The body fluids of the animal, such as serum or ascites fluid, can then be tapped to provide gene product in high concentration. The individual cell lines could also be cultured in vitro, where the bio-pharmaceutical product(s) (including gene product(s)) are naturally secreted into the culture medium from which they can be readily obtained in high concentrations.

For secreted restored bio-pharmaceutical product(s) (including gene product(s)), the culture medium in which the hybrid cells in this invention are cultured can then be assayed for the presence of bio-pharmaceutical product(s). Preferably, the binding specificity of the bio-pharmaceutical product(s) produced by the hybrid cells in this invention is determined by immunoprecipitation or by an in vitro binding assay, such as radioimmunoassay (RIA) or more preferred enzyme-linked immunoabsorbent assay (ELISA). Such techniques and assays are known in the art. The binding affinity of the gene product can, for example, be determined by the Scatchard analysis of Munson and Pollard, Anal. Biochem., 107:220 (1980).

It should be noted that the bio-pharmaceutical product(s) may be used in methods known within the art relating to their localization and/or quantitation (e.g., for use in measuring levels of the protein within appropriate physiological samples, for use in diagnostic methods, for use in imaging the protein, and the like). Bio-pharmaceutical product(s) which possess a protein binding domain, are utilized as pharmacologically-active compounds (“Therapeutics”).

Methodologies which are well-known within the art (e.g., immunoassays, nucleic acid hybridization assays, biological activity assays, and the like) may be used to determine whether one or more restored bio-pharmaceutical product(s) (including restored bio-pharmaceutical product(s)-complexes) are present at either increased or decreased levels, or are absent, within samples derived from patients suffering from a particular disease or disorder, or possessing a predisposition to develop such a disease or disorder, as compared to the levels in samples from subjects not having such disease or disorder or predisposition thereto.

### REFERENCES

Allegrucci, C, Denning, C, Priddle, H, Young, L, Stem-cell consequences of embryo epigenetic defects, Lancet 2004; 364: 206-208.
Alvarez-Dolado M, Pardal R, Garcia-Verdugo J M, Fike J R, Lee H O, Pfeffer K, Lois C, Morrison S J, Alvarez-Buylla A. Fusion of bone-marrow-derived cells with Purkinje neurons, cardiomyocytes and hepatocytes. Nature. 2003; 425: 968-73.
Balsam, L B, Wagers, A J, Christensen, J L, Kofidis, T, Weissman, I L, and Robbins, R C (2004) Haematopoietic stem cells adopt mature haematopoietic fates in ischaemic myocardium, Nature 428: 668-673.
Barberi T, Willis L M, Socci N D and Studer L, (2005) Derivation of Potential Mesenchymal Precursors from Human Embryonic Stem Cells, PLOS Medicine 2(6): 554-560.
Basta, G L, Racanicchi, L, Mancuso, F, Guido, L, Macchiarulo, G, Luca, G, Calabrese, G, Brunetti P, and Calafiore R, (2004) Pancreas transplantation; Neonatal pig pancreatic duct-derived insulin-producing cells: preliminary in vitro studies, Transplantation Proceedings 36; 609-611.
Berry M N, Friend D S. High-yield preparation of isolated rat liver parenchymal cells: a biochemical and fine structural study. J. Cell Biol. 1969; 43: 506-520.
Brandhorst H, Brandhorst D, Brendel M D, Hering B J, Bretzel R G. Assessment of intracellular insulin content during all steps of human islet isolation procedure. Cell Transplant 1998; 7: 489-495.
Bugliani, M, Lupi, R, Del Guerra, S, Boggi, U, Marselli, L, Sbrana, S, Vistoli, F. Torri, S. Del Chiaro, M. Signori, S. Filipponi, F. Del Prato, S. Campa, M. Corsini, V. Campatelli, A. Di Candio, F. Mosca, F. and Marchetti, P. An Alternative and Simple Method to Consistently Prepare Viable Isolated Human Islets for Clinical Transplantation, Transplantation Proceedings, 36: 605-606 (2004).
Carpenter, M K, Cui, X, Hu, Z Y, Jackson, J, Sherman, S, Seiger, A and Wahlberg, L U (1999) Exp. Neurol. 158: 265-278.
Cellesia, F, Tirellia, N, and Hubbell, J A, (2004) Towards a fully-synthetic substitute of alginate: development of a new process using thermal gelation and chemical cross-linking, Biomaterials 25: 5115-5124.
Chang D C, Cell Fusion and Cell Poration by Pulsed Radiofrequency Electric Fields, Electroporation and Electrofusion in Cell Biology, 14: 215-227 (1989).
Cheng T-H, Chang C-R, Joy P, Yablok S, and Gartenberg M R, Controlling Gene Expression in Yeast by Inducible Site-specific Recombination, Nucleic Acids Res. 28(24):E108, 2000.
Chung Y G, Mann M R, Bartolomei M S, and Latham K E, Nuclear-cytoplasmic “tug of war” during cloning: effects of somatic cell nuclei on culture medium preferences of preimplantation cloned mouse embryos, Biol. Reprod. 66: 1178-1184 (2002).
Cogle, C R, Yachnis, A T, Laywell, E D, Zander, D S, Wingard, J R, Steindler, D A, and Scott E W, Bone marrow transdifferentiation in brain after transplantation: a retrospective study. Lancet 2004; 363: 1432-1437.
Crain B J, Tran S D, Mezey E., Transplanted human bone marrow cells generate new brain cells, J Neurol Sci. 2005 Jun. 15; 233(1-2):121-3.
Darnfors C, Flodin A, Andersson K, Caisander G, Lindqvist J, Hyllner J, Wahistrom J, Sartipy P, High-resolution analysis of the subtelomeric regions of human embryonic stem cells. Stem Cells. 2005 April; 23(4):483-488.
Do J T and Scholer H R, Nuclei of embryonic stem cells reprogram somatic cells, Stem Cells. 2004; 22(6):941-9.
Eisenberg L M, Eisenberg C A, Stem cell plasticity, cell fusion, and transdifferentiation. Birth Defects Res Part C Embryo Today. 2003; 69: 209-218.
Erdo F, Trapp T, Buhrle C, Fleischmann B, Hossmann K A, Embryonic stem cell therapy in experimental stroke: host-dependent malignant transformation, Orv Hetil. 2004 Jun. 20; 145(25):1307-13.
Freshney, R I, Culture of Animal Cells, A Manual of Basic Technique, Wiley-Liss, New York, 4th Ed, 2000, pp. 89-120.
Fuchs, S, Satler, L F, Kornowski, R, Okubagzi, P, Weisz, G, Baffour, R, Waksman, R, Weissman, N J, Cerqueira, M, Leon, M B, Epstein, S E, Myocardial Cell Transplantation, Catheter-Based Autologous Bone Marrow Myocardial Injection in No-Option Patients With Advanced Coronary Artery Disease, A Feasibility Study, J. Am. Coll. Cardiol. 2003; 41: 1721-1724.
Gao, S, and Latham K E, Maternal and environmental factors in early cloned embryo development, Cytogenet. Genome Res. 105: 279-284 (2004).
Gardner, D. K. et al., Fertil. Steril. 69: 84 (1998).
Gefter et al, A simple method for polyethylene glycol-promoted hybridization of mouse myeloma cells, Somatic Cell Genet, 3: 231-236, 1977.
Genetic Engineering News Vol 25, Number 7, page 40, Apr. 1, 2005.
Goding, In: Monoclonal Antibodies: Principles and Practice, 2nd Edition, Academic Press, Orlando, Fla., pp. 71-74, 1986.
Grove J E, Bruscia, E, and Krause, D S, Stem Cell Concise Review, Plasticity of Bone Marrow-Derived Stem Cells (2004) Stem Cells 22: 487-500.
Gu, G, Wells, J M, Dombkowski, D, Preffer F, Aronow, B, and Melton, D A, (2004) Global expression analysis of gene regulatory pathways during endocrine pancreatic development, Development 131, 165-179.
Gurol, A O, Yillar, G, Kursun, A O, Kucuk, M, Deniz, G, Aktas, E, Oncan, N and Yilmaz, M T, A Modified Automated Method for Isolation of Viable Pancreatic Islets in Laboratory Animals, Transplantation Proceedings, 36, 1526-1527 (2004).
Hart C, Drewel D, Mueller G, Grassinger J, Zaiss M, Kunz-Schughart L A, Andreesen R, Reichle A, Holler E, Hennemann B. Expression and function of homing-essential molecules and enhanced in vivo homing ability of human peripheral blood-derived hematopoietic progenitor cells after stimulation with stem cell factor. Stem Cells. 2004; 22: 580-589.
Hematti P, Sloand E M, Carvallo, C A et al, Absence of donor-derived keratinocyte stem cells in skin tissues cultured from patients after mobilized peripheral blood hematopoietic stem cell transplantation Experimental Hematology 30 (2002) 943-949.
Humpherys D, Eggan K, Akutsu H, Hochedlinger K, Rideout W M 3rd, Biniszkiewicz D, Yanagimachi R, Jaenisch R. (2001) Epigenetic instability in ES cells and cloned mice. Science 293; 95-97.
Humpherys D, Eggan K, Akutsu H, Friedman A, Hochedlinger K, Yanagimachi R, Lander E S, Golub T R, and Jaenisch R (2002) Abnormal gene expression in cloned mice derived from embryonic stem cell and cumulus cell nuclei. PNAS 99; 12889-12894.
Hussain, M A, Theise, N D, Stem-cell therapy for diabetes mellitus, Lancet 2004; 364: 203-205.
Ishii, S, Sato, Y, Terashima, M, Saito, T. Suzuki, S. Murakami, S, and Gotoh M, (2004) A Novel Method for Determination of ATP, ADP, and AMP Contents of a Single Pancreatic Islet Before Transplantation, Transplantation Proceedings, 36; 1191-1193.
Joshi S N, and Venugopalan P, Experience with NTBC therapy in hereditary tyrosinaemia type I: an alternative to liver transplantation, Ann. Trop. Paediatr. 2004; 24: 259-265.
Joyce N C, Zhu C. Human Corneal Endothelial Cell Proliferation: Potential for Use in Regenerative Medicine. Cornea, 2004; 23: S8-S19.
Kalyani A, Hobson, K, and Rao M S, (1997) Neuroepithelial stem cells from the embryonic spinal cord: isolation, characterization, and clonal analysis, Dev Biol 186: 202-223.
Kang, H-J, Kim, H-S, Zhang, S-Y, Park, K-W, Cho, H-J, Koo, B-W, Kim, Y-J, Lee, D S, Sohn, D-W, Han, K-S, Oh, B-H, Lee, M-M, Park, Y-B, Effects of intracoronary infusion of peripheral blood stem-cells mobilised with granulocyte-colony stimulating factor on left ventricular systolic function and restenosis after coronary stenting in myocardial infarction: the MAGIC cell randomised clinical trial, Lancet 2004; 363: 751-756.
Kim, S U, Human neural stem cells genetically modified for brain repair in neurological disorders, Neuropathol 2004; 24: 159-171.
Kohler and Milstein, Continuous cultures of fused cells secreting antibody of predefined specificity, Nature, 256: 495-497, 1975.
Kohler and Milstein, Derivation of specific antibody-producing tissue culture and tumor lines by cell fusion, Eur. J. Immunol, 6: 511-519, 1976.
Korbutt G S, Elliott J F, Ao Z, Smith D K, Warnock G L, Rajotte R V. Large scale isolation, growth, and function of porcine neonatal islet cells. J Clin Invest 1996; 97: 2119-2129.
Lakey J R, Warnock G L, Rajotte R V, Suarez-Alamazor M E, Ao Z, Shapiro A M, Kneteman N M. Variables in organ donors that affect the recovery of human islets of Langerhans. Transplantation. 1996; 61: 1047-53.
Lakey J R, Warnock G L, Shapiro A M, et al. Intraductal collagenase delivery into the human pancreas using syringe loading or controlled perfusion. Cell Transplantation 1999; 8:285.
Lefkovits, I. and Waldmann, H. (1999) Limiting Dilution Analysis of Cells of the Immune System (Oxford Univ. Press, Oxford).
Levesque, M F, testimony on Adult Stem Cell Research, given at a Senate Science, Technology, and Space Hearing Jul. 14 2004, http://commerce.senate.gov/hearings/testimony.cfm?id=1268&wit_id=3670
Levine, F and Mercola, M (2004) Clinical implications of basic research; No Pancreatic Endocrine Stem Cells? N. Engl. J. Med. 351: 1024-1026.
Lindvall, O (2001) Parkinson's Disease, Stem cell transplantation, The Lancet Supplement 358: S47.
Liu, Y, Merlin, D, Burst, S L, Pochet, M, Madara, J L, and Parkos C A, The Role of CD47 in Neutrophil Transmigration. Increased Rate of Migration Correlates with Increased Cell Surface Expression of CD47, J. Biol. Chem., 276, 40156-40166, 2001.
Love, R, Stem-cell transplantation hope for Parkinson's disease treatment? The Lancet 359: 138, 2002.
Luo Y, Cai J, Ginis 1, Sun Y, Lee S, Yu S X, Hoke A, Rao M. Designing, testing, and validating a focused stem cell microarray for characterization of neural stem cells and progenitor cells. Stem Cells. 2003; 21: 575-87.
Marchetti P, Lupi R, Federici M, Marselli L, Masini M, Boggi U, Del Guerra S, Patane G, Piro S, Anello M, Bergamini E, Purrello F, Lauro R, Mosca F, Sesti G, Del Prato S, Insulin secretory function is impaired in isolated human islets carrying the Gly(972)-->Arg IRS-1 polymorphism, Diabetes 2002; 51: 1419-1424.
Mathur, A, and Martin, J F, Stem cells and repair of the heart, Lancet 2004; 364: 183-192.
Matsumoto S, Kandaswamy R, Sutherland D, et al, Clinical Application of the Two-Layer (University of Wisconsin Solution/Perfluorochemical Plus O.sub.2) Method of Pancreas Preservation Before Transplantation. Transplantation 70: 771-774, 2000.
Matsumoto S, Qualley S, Goel S, et al, Effect of the two-layer (University of Wisconsin solution-perfluorochemical plus O.sub.2) method of pancreas preservation on human islet isolation, as assessed by the Edmonton Isolation Protocol. Transplantation, 74: 1414-1419, 2002.
Matsumoto S, Zhang G, Qualley S, CleverJ, Tombrello Y, Strong D M, Reems J A. The effect of two-layer (University of Wisconsin solution/perfluorochemical) preservation method on clinical grade pancreata prior to islet isolation and transplantation. Transplant Proc. 2004; 36: 1037-1039.
Matsumoto S, Zhang G, Qualley S, Clever J, Tombrello Y, Strong D M, Reems J A. Analysis of donor factors affecting human islet isolation with current isolation protocol. Transplant Proc. 2004; 36: 1034-1036.
Medvinsky A and Smith A, (2003) Stem cells: Fusion brings down barriers. Nature 422, 823-825.
Messina E, De Angelis L, Frati G, Morrone S, Chimenti S, Fiordaliso F, Salio M, Battaglia M, Latronico M V, Coletta M, Vivarelli E, Frati L, Cossu G, Giacomello A. Isolation and Expansion of Adult Cardiac Stem Cells From Human and Murine Heart. Circ Res. 2004 Oct. 7 [Epub ahead of print].
Murry, C E, Soonpaa, M H Reinecke, H, Nakajima, H, Nakajima, H O, Rubart, M, Pasumarthi, K B S, Virag, J I, Bartelmez, S H, Poppa, V, Bradford, G, Dowell, J D, Williams D A and Field L J (2004) Haematopoietic stem cells do not transdifferentiate into cardiac myocytes in myocardial infarcts, Nature 428: 664-668.
Nagler, A, Korenstein-Ilanb, A, Amielc, A, and Avivib L, Granulocyte colony-stimulating factor generates epigenetic and genetic alterations in lymphocytes of normal volunteer donors of stem cells, Exp. Hematol. 32 (2004) 122-130.
Nakamura K, Inaba M, Sugiura K, Yoshimura T, Kwon A H, Kamiyama Y, Ikehara S. Enhancement of allogeneic hematopoietic stem cell engraftment and prevention of GVHD by intra-bone marrow bone marrow transplantation plus donor lymphocyte infusion. Stem Cells 2004; 22: 125-34.
Nygren J M, Jovinge S, Breitbach M, Sawen P, Roll W, Hescheler J, Taneera J, Fleischmann B K, Jacobsen S E, Bone marrow-derived hematopoietic cells generate cardiomyocytes at a low frequency through cell fusion, but not transdifferentiation. Nat Med. 2004; 10: 494-501.
Ohkohchi, N, Itagaki, H, Doi, H, Taguchi, Y, Satomi, S, and Satoh, S, New Technique for Producing Hybridoma by Using Laser Radiation Lasers Surg. Med. 27: 262-268, 2000.
O'Malley, K, and Scott, E W, Stem cell fusion confusion, Exp. Hematol. 32 (2004) 131-134.
Orner, B P, Derda, R, Lewis, R L, Thomson, J A, and Kiessling, L L, Arrays for the Combinatorial Exploration of Cell Adhesion, J. Am. Chem. Soc., 126: 10808-10809, 2004.
Overturf K, Al-Dhalimy M, Finegold M, Grompe M. The repopulation potential of hepatocyte populations differing in size and prior mitotic expansion. Am J Pathol. 1999; 155:2135-2143.
Perin, E C, Dohmann, H F R, Borojevic, R, Silva, S A, Sousa, A L S, Mesquita, C T, Rossi, M I D, Carvalho, A C, Dutra, H S, Dohmann, H J F, Silva, G V, Belem, L, Vivacqua, R, Rangel, F O D, Esporcatte, R, Geng, Y J, Vaughn, W K, Assad, J A R, Mesquita, E T, and Willerson, J T (2003) Transendocardial, Autologous Bone Marrow Cell Transplantation for Severe, Chronic Ischemic Heart Failure. Circulation 107: 2294-2302.
Piper H M, and Isenberg I, Isolation cardiomyocytes (CRC, Boca Raton, 1989).
Poliakova, L, Pirone, A, Farese, A, MacVittie T, and Farney A, (2004) Presence of nonhematopoietic side population cells in the adult human and nonhuman primate pancreas. Transplantation Proceedings 36; 1166-1168.
Raff, M, Adult Stem Cell Plasticity: Fact or Artifact? Annu. Rev. Cell Dev. Biol. 2003, 19:1-22.
Rahman, T M., Selden, C, Khalil, M, Diakanov, I, and Hodgson, H J F, (2004) Alginate-encapsulated Human Hepatoblastoma Cells in an Extracorporeal Perfusion System Improve Some Systemic Parameters of Liver Failure in a Xenogeneic Model. Artificial Organs 28 (5) 476.
Rice, C M and Scolding, N J, Adult stem cells; reprogramming neurological repair? Lancet 2004; 364: 193-199.
Ricordi C, Lacy P E, Finke E H, et al. Automated method for human pancreatic islets. Diabetes 1988; 37: 413.
Ricordi C, Lacy P E and Scharp, D W (1989) Automated islet isolation from human pancreas, Diabetes, 38 Suppl 1: 140-142.
Ricordi C, Gray D W R, Hering B J, et al. Islet isolation assessment in man and large animals. Acta Diabetol Lat 1990; 27: 185.
Romanov Y A, Svintsitskaya V A, Smirnov V N. Searching for alternative sources of postnatal human mesenchymal stem cells: candidate MSC-like cells from umbilical cord. Stem Cells. 2003; 21: 105-110.
Rubio D, Garcia-Castro J, Martin M C, de la Fuente R, Cigudosa J C, Lloyd A C, Bernad A. Spontaneous human adult stem cell transformation, Cancer Res. 2005 Jun. 1; 65(11):4969.
Ruel-Garie'py E and Leroux J-C (2004) In situ-forming hydrogels; review of temperature-sensitive systems, European Journal of Pharmaceutics and Biopharmaceutics 58; 409-426.
Ryan E A, Lakey J R, Rajotte R V, et al. (2001) Clinical outcomes and insulin secretion after islet transplantation with the Edmonton protocol. Diabetes 50: 710.
Schneider, S, Feilen, P J, Slotty, V, Kampfner, D, Preuss, S, Berger, S, Beyer, J, and Pommersheim R, (2001) Multilayer capsules: a promising microencapsulation system for transplantation of pancreatic islets, Biomaterials 22; 1961-1970.
Seaberg R M, Smukler S R, Kieffer T J, Enikolopov G, Asghar Z, Wheeler M B, Korbutt G, van der Kooy D. Clonal identification of multipotent precursors from adult mouse pancreas that generate neural and pancreatic lineages. Nat Biotechnol. 2004; 22: 1115-24. Epub 2004 Aug. 22.
Seshagiri P B et al, 1993, Am J Primatol 29; 81-91.
Shapiro, A. M. J., Jonathan, R. T M. B et al. (2000) Islet Transplantation in Seven Patients with Type I Diabetes Mellitus Using a Glucocorticoid-Free Immunosuppressive Regimen. N Eng J Med 343; 230-238.
Solter D and Knowles B B, Proc. Natl. Acad. Sci. U.S.A. 75, 5565 (1978)
Sutovsky P, and Prather R S, (2004) Nuclear remodeling after SCNT: a contractor's nightmare, Trends in Biotechnol. 22: 205.
Tamaki S, Eckert K, et al, (2002) J Neurosci Res 69: 1976-1986.
Theise, N D, Krause, D S, and Sharkis S (2003) Comment on “Little Evidence for Developmental Plasticity of Adult Hematopoietic Stem Cells”, Science 299: 1317a.
Thomson, J A, et al., Proc. Natl. Acad. Sci. U.S.A. 92, 7844 (1995).
Thompson J A, Itskowitz-Eldor J, Shapiro S S, Waknitz M A, Swiergiel J J, Marshall V S, and Jones J M, Embryonic Stem Cell Lines Derived from Human Blastocytes, Science 282: 1145-1147, 1998.
Tomczuk M, Takahashi Y, Huang J, Murase S, Mistretta M, Klaffky E, Suthernland A, Boiling L, Coonrod S, Marcinkiewicz C, Sheppard D, Stepp M A and White J, role of multiple beta-1 integrins in cell adhesion to the disintegrin domains of ADAMs 2 and 3, Exper Cell Res 290: 68-81, 2003.
Trevor, K T, Cover, C, Ruiz, Y W, Akporiaye, E T, Hersh, E M, Landais, D, Taylor, R R, King, A D, and Walters, R E (2004) Generation of dendritic cell-tumor cell hybrids by electrofusion for clinical vaccine application Cancer Immunology, Immunother. 53: 705-714.
Troupepe V, et al, 1999, Dev Biol 208: 166-188.
Turnpenny L, Brickwood S, Spalluto C M, Piper K, Cameron I T, Wilson D I, Hanley N A. Derivation of human embryonic germ cells: an alternative source of pluripotent stem cells. Stem Cells. 2003; 21: 598-609.
Uchida N, et al, 2000 Proc Natl Acad Sci, U.S.A. 97; 14720-14725.
Vassilopoulos G, Wang, P-R, Russell, D W (2003) Transplanted bone marrow regenerates liver by cell fusion, Nature 422: 901-904.
Vidricaire G, Jardine, K, and McBurney, M W (1994) Expression of the Brachyury gene during mesoderm development in differentiating embryonal carcinoma cell cultures, Development 120: 115-122.
Wang J, Song, L J, Gerber, D A, Fair, J H, Rice, L, LaPaglia, M, and Andreoni K A, (2004) A Model Utilizing Adult Murine Stem Cells for Creation of Personalized Islets for Transplantation, Transplantation Proceedings, 36, 1188-1190.
Wang X, Willenbring H, Akkari Y, Torimaru Y, Foster M, Al-Dhalimy M, Lagasse E, Finegold M, Olson S and Grompe M (2003) Cell Fusion is the Principal Source of Bone Marrow-Derived-Hepatocytes, Nature 422: 897-901.
Weaver V M, et al, (1997) J. Cell Biol. 137; 231-245.
Weimann J M, Johansson C B, Trejo A and Blau H M, (2003) Stable reprogrammed heterokaryons form spontaneously in Purkinje neurons after bone marrow transplant, Nature Cell Biology 5: 959.
Wobus A M, Boheler K R, Embryonic stem cells: prospects for developmental biology and cell therapy. Physiol Rev. 2005 April; 85(2):635-78.
Wollert, K C, Meyer, G P, Lotz, J, Ringes-Lichtenberg, S, Lippolt, P, Breidenbach, C, Fichtner, S, Korte, T, Hornig, B, Messinger, D, Arseniev, L, Hertenstein, B, Ganser, A, and Drexler, H, Intracoronary autologous bone-marrow cell transfer after myocardial infarction: the BOOST randomised controlled clinical trial, Lancet 2004; 364: 141-148.
Wu, X, Walker, J, Zhang, J, Ding, S, and Schultz, P G, (2004) Purmorphamine Induces Osteogenesis by Activation of the Hedgehog Signaling Pathway, J. Chem. Biol.; 11(9): 1229-1238.
Ye, M, Iwasaki, H, Laiosa, C V, Stadtfeld, M, Xie H, Heck, S, Clausen B, Akashi, K, and Graf, T, (2003) Hematopoietic Stem Cells Expressing the Myeloid Lysozyme Gene Retain Long-Term, Multilineage Repopulation Potential, Immunity 19: 689-699.
Young, W, abstract posted Oct. 13, 2004 for conference, 4th Annual Stem Cells & Regenerative Medicine Oct. 18-19, 2004, Strategic Research Institute, Prinston, N.J., Westin Prinston at Forrestal Village, web address, www.srinstitute.com/ApplicationFiles/web/WebFrame.cfm?web_id=225&webpageid=2421 & prioritycode=DEM004875
Zeng, X, Miura, T, Luo, Y, Bhattacharya, B, Condie, B, Chen, J, Ginis, I, Lyons, I, Mejido, J, Puri, R K, Rao, M S, Freed, W J (2004) Properties of Pluripotent Human Embryonic Stem Cells BG01 and BG02, Stem Cells 22: 292-312.
Zentner, G M, et al, (2001) Biodegradable block copolymers for delivery of proteins and water-insoluble drugs, J. Control. Release 72; 203-215.

What is claimed is:

1. A method of generating hybrid cell mixtures, hybrid cells, or hybrid cell lines in vitro that are of either mammalian origin alone or avian origin alone, comprising the steps of:
(a) forming a mixture of (i) transit amplifying cells or terminally differentiated somatic cells or both, (hereby collectively termed “LCSO cells”) with (ii) SC cells by mixing said LCSO cells with said SC cells, said SC cells chosen from a group consisting of nucleated adult stem cells, nucleated stem cell-like cells, and nucleated transit amplifying cells;
(b) forming hybrid cells by fusing cells from said mixture of LCSO cells and SC cells, said hybrid cells hereby termed “LCSOSC cells”, whereas said LCSOSC cells, LCSO cells and SC cells, or subset(s) containing hybrid cells therefrom hereby collectively termed “collective SC mixture”, and optionally comprising selecting said LCSOSC cells from collective SC mixture hereby termed “selected LCSOSC cells”;
(c) propagating said selected LCSOSC cells or said LCSOSC cells within collective SC mixture, and optionally comprising performing any of the following step(s) in any order, including repetition of an optional step for the said collective SC mixture or said selected LCSOSC cells;
(c1) cytologically examining;
(c2) monitoring for one or more bio-pharmaceutical products;
(c3) propagating;
(c4) generating a tissue therefrom;
(c5) generating an organ therefrom;
(c6) generating neurospheres therefrom; and
(c7) adding “coaxing factor(s)”.

2. The method according to claim 1, wherein the said LCSO and SC cells are restricted to mammalian origin.

3. The method according to claim 2, wherein the said LCSO cells are restricted to terminally differentiated somatic cells.

4. The method according to claim 2, wherein the said SC cells are restricted to non-immortalized cells.

5. The method according to claim 2, wherein the fusion step comprises being conducted by conventional fusion technique(s), by electrical pulse(s), by laser pulse(s), by radiofrequency pulse(s), by natural fusion technique(s) or combinations thereof in any order.

6. The method according to claim 2, wherein the fusion step is conducted by conventional fusion technique(s).

7. The method according to claim 2, wherein the fusion step is conducted by electroporation(s).

8. The method according to claim 2, wherein the fusion step is conducted by radiofrequency electrical pulses.

9. The method according to claim 2, wherein the fusion step is conducted by laser pulse(s).

10. The method according to claim 2, wherein the fusion step is conducted by natural fusion technique(s).

11. The method according to claim 2, wherein the said LCSO cells are from mesoderm.

12. The method according to claim 2, wherein the said LCSO cells are from ectoderm.

13. The method according to claim 2, wherein the said LCSO cells are from endoderm.

14. The method according to claim 2, wherein the said LCSO cells are chosen from the group consisting of biliary origin, bone marrow origin, endocrine origin, endodermal origin, epidermal origin, exocrine origin, gut origin, hematopoietic origin, hepatic origin, intestinal origin, mesenchymal origin, musculoskeletal origin, neural origin, neuroendocrine origin, neuronal origin, ophthalmic origin, pancreatic origin, placental origin, pulmonary origin, renal origin, salivary gland origin, smooth muscular origin, striated muscular origin and vascular origin.

15. The method according to claim 2, wherein the said LCSO cells are further restricted within the population of the following groups of cells comprising; pancreatic cells, pancreatic islet cells, pancreatic beta cells, pancreatic alpha cells, or pancreatic delta cells, or any combination thereof; cardiomyocytes; or neuronal cells, neuroglial cells, oligodendrocytes, or any combination thereof.

16. The method according to claim 2, wherein the said LCSO cells are further restricted within the population of the following group of cells comprising; pancreatic cells, pancreatic islet cells, pancreatic beta cells, pancreatic alpha cells, or pancreatic delta cells, or combination thereof.

17. The method according to claim 2, wherein the said SC cells are comprised of adult stem cells.

18. The method according to claim 2, wherein the said SC cells are further restricted within the population of the following group of cells comprising; bone marrow cells, hematopoietic progenitor cells, HSC, MSC, and optionally excludes polymorphonuclear cells.

19. The method according to claim 16, wherein the said SC cells are further restricted within the population of the following group of cells; bone marrow cells or hematopoietic progenitor cells.

20. The method according to claim 15, wherein the fusion step is conducted by conventional fusion technique(s), by electrical pulse(s), by laser pulse(s), by radiofrequency pulse(s), by natural fusion technique(s) or combinations thereof in any order.

21. The method according to claim 1, wherein the LCSO cells are monitored for restorage of damage or for at least one defective, dysfunctional, underexpressed or lost bio-pharmaceutical product or of bio-pharmaceutical products previously not expressed in LCSO cells prior to their fusion, and optionally comprising performing any of the following step(s) in any order, including repetition of an optional step or substep for the said collective SC mixture or said selected LCSOSC cells;
(a) preparing a tissue by generating a tissue from cell growth of the said collective SC mixture or said selected LCSOSC cells, and optionally comprising a substep of adding additional cells or cellular-positioning engineering within the tissue being prepared or both in tissue preparation;
(b) preparing an organ by generating an organ from cell growth of one or more of the prepared tissue, said collective SC mixture or said selected LCSOSC cells, and optionally comprising a substep of adding additional cells or cellular-positioning engineering within the organ being prepared or both in organ preparation; and
(c) preparing neurospheres by generating neurospheres from cell growth of the said collective SC mixture or said selected LCSOSC cells, and optionally comprising a substep of adding additional cells or cellular-positioning engineering within the neurospheres being prepared or both in neurosphere preparation.

22. The method according to claim 21, further comprising the step of placing the said collective SC mixture, said selected LCSOSC cells, said prepared tissue, said prepared organ or said neurospheres within or on a mammal or aves.

23. The method according to claim 22, wherein the placement is restricted to mammals of the same genus and species.

24. The method according to claim 23, wherein the placement is post-partum.

25. The method according to claim 23, wherein the placement is pre-partum.

26. The method according to claim 23, whereas the placement is within an allogeneic species.

27. The method according to claim 23, whereas the placement is within a syngeneic species.

28. The method according to claim 21, further comprising a step for “LCSOSC hybrid cell conditioning” before or after the fusion step.

29. The method according to claim 28, further comprising the step of placing the said collective SC mixture, said selected LCSOSC cells, said prepared tissue, said prepared organ or said neurospheres within or on a mammal or aves.

30. The method according to claim 29, wherein the placement is restricted to mammalian origin.

31. The method according to claim 30, wherein the said LCSO cells are further restricted within the population of the following groups of cells comprising; pancreatic cells, pancreatic islet cells, pancreatic beta cells, pancreatic alpha cells, or pancreatic delta cells, or combination thereof; cardiomyocytes; or neuronal cells, neuroglial cells, oligodendrocytes, or any combination thereof.

32. The method according to claim 30, wherein the said SC cells are further restricted within the population of the following group of cells comprising; bone marrow cells, hematopoietic progenitor cells, HSC, MSC, and optionally excludes polymorphonuclear cells.

33. The method according to claim 30, wherein the fusion step comprises being conducted by conventional fusion technique(s), by electroporation(s), by radiofrequency electrical pulse(s), by laser pulse(s), by natural fusion technique(s) or combinations thereof in any order.

34. The method according to claim 30, wherein said “LCSOSC hybrid cell conditioning” comprising said selected LCSOSC cells, said collective SC mixture, said tissues, said organs, said neurospheres or combination thereof are placed within or on a mammal of the same genus and species.

35. The method according to claim 34, wherein the placement is post-partum.

36. The method according to claim 34, whereas the placement is within an allogeneic or syngeneic species.

37. A method of generating hybrid cell mixtures, hybrid cells, or hybrid cell lines in vitro that are of either mammalian origin alone or avian origin alone, comprising the steps of:
(a) forming a mixture of (i) nonlymphocytic LCSO cells” herein termed “NL-LCSO cells” with (ii) nucleated ES or nucleated EG cells herein collectively termed “ESG cells”, by mixing said NL-LCSO cells with said ESG cells;
(b) forming hybrid cells by fusing cells from said mixture of NL-LCSO cells and ESG cells, said hybrid cells hereby termed “NL-LCSO-ESG cells”, whereas said NL-LCSO-ESG cells, NL-LCSO cells and ESG cells, or subset(s) containing hybrid cells therefrom hereby collectively termed “collective ESG mixture”, and optionally comprising selecting said NL-LCSO-ESG cells collective ESG mixture therefrom hereby termed “selected NL-LCSO-ESG cells”;
(c) propagating said selected NL-LCSO-ESG cells or propagating said NL-LCSO-ESG cells within said mixture of NL-LCSO cells and ESG cells or; and optionally comprising performing any of the following step(s) in any order, including repetition of an optional step for the said collective ESG mixture or said selected NL-LCSO-ESG cells;
(c1) cytologically examining;
(c2) monitoring for one or more bio-pharmaceutical products;
(c3) propagating;
(c4) generating a tissue therefrom;
(c5) generating an organ therefrom;
(c6) generating neurospheres therefrom; and
(c7) adding “coaxing factor(s)”.

38. The method according to claim 37, further comprising a step for “NL-LCSO-ESG hybrid cell conditioning” before or after the fusion step.

39. The method according to claim 38, further comprising the step of placing the said collective SC mixture, said selected LCSOSC cells, said generated tissue, said generated organ or said neurospheres within or on a mammal or aves.

40. The method according to claim 39, wherein the said NL-LCSO cells are further restricted to nonlymphoid cells.

41. The said LCSOSC cells, said propagated LCSOSC cells, said hybrid cell lines resulting from propagation of said LCSOSC cells, tissues, organs or neurospheres generated therefrom or any combination thereof.
